(19) United States
(12) Patent Application Publication
(10) Pub. No.: US 2021/0009548 A1
(43) Pub. Date: Jan. 14, 2021

(54) BURST ATOMIZATION FRACTIONATION SYSTEM, METHOD AND APPARATUS

(71) Applicant: FOG ATOMIC TECHNOLOGIES LLC, Medina, OH (US)

(72) Inventors: FRANK PAPA, Medina, OH (US); MARION MCEUEN, Tucson, AZ (US); JEFFREY F. VOGLER, Tucson, AZ (US); THOMAS B. CASSERLY, San Ramon, CA (US)

(73) Assignee: FOG ATOMIC TECHNOLOGIES LLC, Medina, OH (US)

(21) Appl. No.: 16/924,811

(22) Filed: Jul. 9, 2020

Related U.S. Application Data
(60) Provisional application No. 62/872,797, filed on Jul. 11, 2019.

(51) Int. Cl. C07D 311/78; C07C 37/00; B01D 3/10; B01D 3/34; B01D 5/00; B01D 3/32; B01D 1/20

(52) U.S. Cl. CPC C07D 311/78; C07C 37/004; B01D 3/106; B01D 1/20; B01D 5/006; B01D 5/0006; B01D 3/32; B01D 3/343

(57) ABSTRACT

Systems, methods and apparatus for fractioning mixtures comprising combinations of volatiles, essentially non-volatiles and non-volatile solutes are disclosed. Embodiments of a burst atomization fractionation apparatus comprises an atomization chamber into which a liquid mixture is atomized across a pressure gradient. In various embodiments, a mixture for fractionation comprises mixtures of solvents, solvents and oils, used engine oil, or salt water. In various examples, a solute undergoes a chemical transformation during the fractionation process, such as dehydrogenation, dehydration, or decarboxylation.

### CROSS-REFERENCE TO RELATED APPLICATIONS

This application claims priority to and the benefit of U.S. Provisional Patent Application Ser. No. 62/872,797 filed Jul. 11, 2019 and entitled “Burst Atomization Fractionation System, Method and Apparatus,” the disclosure of which is incorporated herein by reference in its entirety for all purposes.

### FIELD

The present disclosure generally relates to fractionation of mixtures, and in particular, to a system, method and apparatus for separating volatile solvent mixtures, volatiles from essentially non-volatiles, volatiles from non-volatile solutes, and for the recovery of petroleum oils, natural products, and solvents.

### BACKGROUND

Various fractionation processes may rely on distillation at ambient pressure, wherein a vessel containing a mixture of substances is heated to boiling to vaporize the volatiles in the mixture and fractionate volatiles having different volatility and/or separate volatiles from nonvolatile materials. For the separation of liquids close in volatility, a fractionating column may be placed between the vaporizing mixture of volatiles and the condenser. The solvent vapors are then condensed back into the liquid phase inside a cooled/jacketed condenser and the condensates collected as liquid fractions. The poorly volatile materials, such as solutes, remain in the vessel as they cannot be volatilized. Simple distillation such as this typically requires a large amount of energy to heat the distillation “pot.” Scale-up to industrial sized systems is limited by the size of the vessel that can be efficiently heated, and the nature of the mixture to be distilled, e.g., if the mixture comprises constituents subject to pyrolytic or other degradations, cis-/trans-alkene isomerization, racemization of chiral centers, or other unwanted reactions.

Simple distillation is also limited to several controllable parameters, e.g., the temperature at which the vessel is heated, fractionation column lengths, temperature and length of the cooling condenser, and the pressure (e.g., a vacuum). Further, unless the distillation system is under vacuum or blanketed by expensive N₂ or Argon, the system may be infiltrated by oxygen from the air, exposing the various constituents in the mixture to possible oxidation reactions.

Given the shortcomings in conventional distillations, particularly when temperature sensitive compounds are present, new fractionation systems, methods and apparatus are still needed. Further, new methods to achieve simultaneous fractionation and natural products chemistry are desired. For example, given the complexity required for viable and sustainable production of commercially useful cannabinoids, new systems and methods are needed to streamline and even combine both extraction solvent recovery and decarboxylation of cannabinoid carboxylic acids.

### SUMMARY

In various embodiments, systems, methods and apparatus for energy efficient fractionation of mixtures are disclosed. Such processes provide efficient separation of (1) volatiles from volatiles, (2) volatiles from essentially non-volatile substances, and (3) volatiles from non-volatile solutes.

In various embodiments, systems, methods and apparatus for separation of liquids close in volatility, such as solvent mixtures, are disclosed. In other aspects, systems, methods and apparatus for separation of volatile solvents from essentially non-volatile oils, such as separation of solvents from used engine and lubricating oil and natural products are disclosed. In other aspect, systems, methods and apparatus for separation of volatile solvents from non-volatile solutes, such as desalination of water, are disclosed. In each of these variations, a goal is recovery and reuse of each component present in a mixture, whether it is a volatile, essentially non-volatile, or non-volatile solute component present in the mixture. In other aspects, materials for commercial sale or reuse are separated from materials destined for waste.

In various embodiments, a liquid mixture for fractionation in accordance with the present disclosure comprises a mixture of volatiles, such as complex mixtures of solvents resulting from various chemical extraction processes. Such complex liquid solvent mixtures may be the result of industrial processes such as extraction of natural isoprene rubber from plant material or resins, degreasing of steel in a foundry, degreasing machine parts in a machine shop, recrystallization of organic compounds in pharmaceutical manufacturing, and so forth.

In various embodiments, a liquid mixture for fractionation in accordance with the present disclosure comprises a mixture of volatiles and materials that are deemed “essentially non-volatile,” like fossil fuel oils and fatty acids. For example, such mixtures may comprise natural products, including tree, nut, or fruit oils, biofuel oils obtained from algae, tree and plant resins, carbohydrates, lignans, and cannabinoid oils from Cannabis, dissolved in one or more volatile solvents. More specific examples include, but are not limited to, corn, canola, or olive oil in hexane, polyisoprene rubber in a mixture of hexanes, pentanes and toluene, and cannabinoid oils in ethanol, hexane or butane. In various aspects, the essentially non-volatile material may be a machine or lubricating oil, such as from a vacuum pump or an engine, and the volatile substances dissolved in the oil may be solvents that were entrained into the oil while the oil was being used in a particular motor or some other purpose. In other words, the oil may be the majority of the mixture, such as in the case of used engine oil containing dissolved solvents, or the minority, such as in the case of cannabinoid oils extracted in ethanol.

In various embodiments, a mixture for fractionation in accordance with the present disclosure comprises an entirely non-volatile solute, like a salt or a metal, including oxides, nitrides thereof, dissolved in a volatile liquid including water. In various examples, the mixture for fractionation comprises salt water. In various aspects, fractionation of salt water comprises a desalination process for desalinating sea water. In other examples, fractionation may comprise separation of an aqueous mixture of metal oxides obtained in an industrial process.

In various embodiments, fractionation of a mixture of natural oils (essentially non-volatile substances) from an extraction solvent or mixtures of solvents further comprises the simultaneous organic reaction of the natural oil. For example, fractionation of a mixture may further comprise affecting decarboxylation of cannabinoid acids and recovery of solvent used to extract the cannabinoid acids from plant material are provided.

In various embodiments, a system for affecting decarboxylation of cannabinoid acids and recovery of extraction solvent comprises a standalone apparatus. In other embodiments, a system for affecting decarboxylation of cannabinoid acids and recovery of extraction solvent comprises an apparatus connected to a wiped film or other fractionation system.

In various embodiments, a fractionation apparatus is provided. In various embodiments, the apparatus comprises: an atomization chamber with an inlet and outlet, further comprising an atomization nozzle or ultrasonic nebulizer at the inlet; a condenser in fluidic communication with the outlet; and a vacuum pump. In use, a pressurized and heated liquid mixture comprising at least one of volatiles, essentially non-volatiles, and non-volatile solutes is atomized through the atomization nozzle. In other embodiments, unpressurized and optionally heated liquid mixture is atomized through an ultrasonic nozzle, into the atomization chamber.

In various embodiments, a feed gas may be used to pressurize a liquid mixture for fractionation prior to atomization. In various embodiments, a disruptor gas affecting the atomization process may also be used. A disruptor gas may comprise CO₂. Both the feed gas and the disrupter gas, particularly if the same substance chemically, may be captured and recycled.

In various embodiments, a decarboxylation and solvent recovery method is provided. The method comprises: pressurizing and heating a liquid mixture to a pressure P1 and temperature T1, the liquid mixture comprising an extraction solvent and a cannabinoid carboxylic acid dissolved, dispersed, or solubilized therein; directing the pressurized and heated liquid mixture to an atomizing nozzle fitted to the inlet of an atomization chamber, wherein the liquid mixture is atomized across a pressure gradient into the atomization chamber maintained at a second pressure P2 that is less than P1 and at a second temperature T2, and wherein the cannabinoid carboxylic acid is decarboxylated to its corresponding cannabinoid; and condensing the cannabinoid in a first condenser C1 maintained at temperature CT1 while pumping the atomized extraction solvent and the CO₂ liberated in the decarboxylation reaction to a second condenser C2 maintained at a temperature CT2 by a vacuum pump, wherein the extraction solvent condenses. In various embodiments of the method, the atomization nozzle may comprise an ultrasonic atomizer (i.e., a nebulizer) run off electricity and tuned to a desired frequency so as to obtain a target particle size from the nebulizer.

In various embodiments, a decarboxylation and solvent recovery apparatus is automated such that the variables, P1, P2, T1, T2, CT1 and CT2, along with other variables such as flow rate through the atomizing nozzle and between apparatus components, are each independently controllable. In various embodiments, a “smart” system is computerized, wherein automatic input, output gas, and liquid sensing provides feedback for the system to self-adjust in order to maintain quality and throughput.

In various embodiments of the method, liquid material condensing in the first condenser C1 may be maintained at temperature CT1 sufficient to allow additional solvent, other volatile materials, and/or CO₂ to continue to evolve from the liquid condensed in C1. In other embodiments, C1 is maintained at a temperature CT1 sufficient to limit or prevent further evolution of volatile materials from the liquid condensed in C1.

In various embodiments of the method, the liquid thus condensed in C1 may be captured at the bottom of the condenser, e.g., in a retaining tank, or removed as a process stream using gravity or liquid pump. The nature of this material, such as comprising a natural products mixture comprising cannabinoid oils, may be analyzed such as by spectroscopic methods and reprocessed. Such reprocessing methods may include repeating the pressurization/atomization. For example, if the captured oil is determined to still contain undesirable amounts of ethanol or is found contaminated with other volatile materials that may have originated from the plant material previously extracted, it can be reintroduced to the feed mixture as a “reflux” or further processed in a second stage.

By controlling P1, P2, T1 and T2, and thus the average particle size of the atomized droplets, the desired cannabinoid components can be accurately and selectively removed from the liquid extraction mixture. By utilizing the large surface area of the atomized droplets, the temperature and the vacuum pressure in the atomization chamber, the amount of energy and heated surface area needed for removal of the extraction solvent, volatiles and CO₂ from a liquid extraction mixture can be significantly reduced by eliminating waste heat and providing efficient heat transfer by maintaining turbulent flow in the atomization chamber. Such a system as disclosed herein is scalable in size, and is not limited to flow restrictions because multiple nozzles can be added in parallel. In various embodiments, the decarboxylation and solvent recovery system herein comprises a smaller footprint than traditional systems for solvent distillation and cannabinoid acid decarboxylation because the system herein does not rely on heated surface area to evaporate extraction solvent, other volatiles and CO₂ from the liquid extraction mixture.

In various embodiments, atomization, vapor condensation and/or decarboxylation of various cannabinoid carboxylic acids produce heat, both from the heat of condensation of the solvents in the liquid extraction mixture when, for example, condensed on a heat exchanger, and heat released from exothermic organic reactions. This heat may be recycled, e.g. through a heat exchanger, to the step of heating the liquid extraction mixture prior to atomization, or to heating the atomization nozzle. Basically, energy that was put into pressurizing and heating a liquid extraction mixture prior to atomization is partly recoverable from the atomization process where a tremendous amount of energy is removed in the atomization.

### BRIEF DESCRIPTION OF THE DRAWING FIGURES

The subject matter of the present disclosure is pointed out with particularity, and claimed distinctly in the concluding portion of the specification. A more complete understanding of the present disclosure, however, may best be obtained by referring to the detailed description and claims when considered in connection with the following drawing figures:

FIG. 1 illustrates various embodiments of a single tank burst atomization fractionation apparatus in accordance with the present disclosure;

FIG. 2 illustrates various embodiments of a multi-tank burst atomization fractionation apparatus in accordance with the present disclosure; and

FIG. 3 illustrates various embodiments of a multi-tank burst atomization fractionation apparatus comprising two condensers, useful for example in the simultaneous cannabinoid decarboxylation and extraction solvent recovery in accordance with the present disclosure.

### DETAILED DESCRIPTION

The detailed description of exemplary embodiments references the accompanying drawings, which show exemplary embodiments by way of illustration and their best mode. While these exemplary embodiments are described in sufficient detail to enable those skilled in the art to practice the invention, it should be understood that other embodiments may be realized and that logical, chemical, and mechanical changes may be made without departing from the spirit and scope of the inventions. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

In various embodiments, systems, methods and apparatus for energy efficient fractionation of mixtures are disclosed. Such processes provide efficient separation of at least (1) mixtures of volatiles; (2) mixtures of volatiles and essentially non-volatile substances; and (3) mixtures of non-volatile solutes and volatiles.

In various embodiments, systems, methods and apparatus for energy efficient fractionation of volatile solvent mixtures are disclosed. Mixtures of at least two volatile solvents for fractionation in accordance with the present disclosure may be an output stream from an industrial process, such as a solvent waste stream from a machining operation.

In various embodiments, systems, methods and apparatus for energy efficient fractionation of volatiles from essentially non-volatile substances are disclosed. Mixtures comprising what are essentially non-volatile substances, such as natural oils, and volatile solvents, may be intermediate extraction mixtures obtained in various chemical processes. In various embodiments, the essentially non-volatile substance may comprise a nut, fruit, plant, flower, tree or seed oil, and the volatile substance may comprise an extraction solvent that was used to extract the oils from the natural nut, fruit, plant, flower, tree or seed material.

The present disclosure is not in any way limited by the nature of the essentially non-volatile substance, the non-volatile solute, or the nature of the solvent in the liquid mixture to be fractionated. In various embodiments, an extraction solvent may be used to extract a natural product of interest from its natural source, and then the fractionation as disclosed herein is used to isolate the natural product and recover the extraction solvent so that it can be reused.

In various embodiments, a system, method and apparatus for energy efficient fractionation of volatiles from non-volatile solutes are disclosed. The non-volatile solutes may include inorganic salts such as sodium chloride (e.g., sea salt), or organic salts (e.g., carboxylate salts).

In various embodiments, a system, method and apparatus for fractionating a liquid extraction mixture and affecting a chemical reaction are provided. The present disclosure provides for fractionating solute from solvent and optionally changing the chemical structure of the solute by a chemical reaction occurring during the fractionation. In various embodiments, the solute is chemically changed by any chemical reaction. These chemical reactions that may occur during the fractionation process include, but are not limited to, dehydrogenation, dehydration, decarboxylation, cis-/trans-isomerization, intramolecular reactions such as Diels-Alder cyclization, and other C—C bond rearrangement reactions. These chemical reactions may be thermally catalyzed, and/or may be catalyzed by certain metals or other catalysts strategically placed within the fractionation apparatus described herein.

In various embodiments of the present disclosure, a system, method and apparatus for affecting both decarboxylation of cannabinoid acids and extraction solvent recovery are provided. A decarboxylation and solvent recovery apparatus may comprise an atomization chamber further comprising an inlet atomization nozzle or ultrasonic nebulizer; a condenser; and a vacuum pump. A system may comprise both a decarboxylation and solvent recovery apparatus, and a method of obtaining cannabinoid oils and recovered solvent using the decarboxylation and solvent recovery apparatus.

In various embodiments of the present disclosure, a method of simultaneously obtaining cannabinoids from cannabinoid carboxylic acids and recovering the extraction solvent previously used to obtain the cannabinoid carboxylic acids from plant material is provided.

### Definitions and Interpretations

As used herein, the term “mixture” refers to a liquid chemical mixture comprising any combination of volatile components, essentially non-volatile components, and non-volatile solutes. Non-limiting examples of volatile components include organic solvents and water, having boiling points at ambient pressure of up to about 200° C. Non-limiting examples of organic volatiles include low molecular weight (C1-C4) alcohols, C5-C10 aliphatic and aromatic hydrocarbons such as pentanes, hexanes, cyclohexane, petroleum ethers, benzene, toluene, o-, m- and p-xylenes, along with low molecular weight volatile carboxylic acids (e.g., formic acid, acetic acid, etc.), ethers (e.g., diethyl ether), diols, esters (e.g., ethyl acetate), and so forth. Non-limiting examples of “essentially non-volatile” components include vegetable oils, terpenes, terpenoids, sesquiterpenes, sesquiterpenoids, and the like, having boiling points at ambient pressure at least greater than about 200° C. Non-limiting examples of non-volatile solutes include inorganic salts (e.g., sodium chloride, potassium chloride, phosphates, sulfates, etc.), amino acids, and organic salts (e.g., sodium carboxylates, etc.).

The temperature suggested here as the temperature cut-off between volatiles and what are referred to as essentially non-volatiles (e.g., 200° C.) is by no means a strict delineation between volatile substances and essentially non-volatile substances. There are other ways to visualize this distinction. For example, another way to understand the distinction is that volatile materials, like low molecular weight solvents, are likely amenable to ambient pressure distillation, whereas essentially non-volatile substances, such as a vegetable oil, would more than likely thermally decompose, at least to some extent, during distillation at ambient pressure. For example, the boiling point of linoleic acid is about 229° C. at a reduced pressure of 16 mmHg. Thus, distillation of linoleic acid at ambient pressure might be doable, but is not likely practical, and so this fatty acid would be considered essentially non-volatile. Another essentially non-volatile material of interest herein is lubricating oil, which depending on its precise composition, has a boiling range of about 370° C. to 600° C. Thus, an exemplary mixture for fractionation in accordance with the present disclosure comprises ethanol (the volatile component) and motor oil (the essentially non-volatile component) in various ratios. In various embodiments, a volatile substance in a mixture for fractionation comprises an extraction solvent (defined below), such as ethanol.

As used herein, the term “atomization” refers to the conversion of a bulk liquid material into very small droplets. The droplet size of an atomized liquid is usually on the scale of nanometers to microns. Sometimes these fine droplets are referred to as “particles.” In effect, atomization is related to aerosolization of a liquid except that the “fog” or “mist” thus produced consists of liquid droplets of very small size. In various embodiments herein, a liquid mixture is atomized into micron- or nanosized particles by way of an atomizing nozzle or an ultrasonic nebulizer. However, it is possible that atomization of a liquid mixture comprising volatile solvents and essentially non-volatile oils will result in a mixture of gasses and aerosols or mists, such as if aerosolized solvent impinges on heated surfaces within an atomization chamber or simply vaporizes from atomized liquid droplets and the essentially non-volatile oils are simply misted. As discussed herein, atomization can be controlled (and thus particle size adjusted) by temperature and pressure of the liquid to be atomized prior to entering the nozzle or nebulizer, the nature of the nozzle or nebulizer, and/or by the pressure (e.g., vacuum) in the atomization chamber (i.e., the pressure drop). In various embodiments, a pump (compressor) or pressurized feed gas tank, may be used to pressurize the liquid at the inlet side of the atomizing nozzle, and/or a vacuum pump may be used to reduce the pressure (i.e., create a vacuum) inside the atomization chamber, thus effectively controlling and maintaining a pressure gradient (drop) for the atomization process.

As used herein, the term “extraction solvent” refers to either water or an organic solvent used to extract various natural products from their natural sources, e.g., cannabinoids from plant material, essential oils from flower pedals, alkaloids from plant leaves or stems, fatty acids from algae, vegetable oils from vegetable sources, and nut oil from nuts. In various embodiments, an extraction solvent may comprise a lower molecular weight alcohol, e.g., methanol, ethanol, n-propanol, isopropanol, n-butanol, and the like, or a lower molecular weight diol, e.g., ethylene glycol, 1,2-propylene glycol, or 1,3-propylene glycol. In other examples, an extraction solvent may comprise an ether, ester, hydrocarbon, aromatic hydrocarbon, chlorinated hydrocarbon, fluorinated hydrocarbon, chlorinated/fluorinated hydrocarbon, and the like, e.g., butanes, pentanes, hexanes, benzene, toluene, xylenes, chloroform, methylene chloride, diethyl ether, ethyl acetate, and so forth. In general, the solvents for use herein will typically have boiling points (at ambient pressure) of ≤100°-150° C. In various embodiments, an extraction solvent for use herein may be chosen on the basis of its ability and/or its selectivity to extract and dissolve natural products of interest, ability to penetrate nut, fruit, plant, aquatic plant, flower, tree bark or seed material, volatility (vapor pressure/boiling point), freezing point, toxicity, stability, cost and other considerations. In various embodiments, a system, method and apparatus described herein provide for recovery of an extraction solvent and the essentially non-volatile substances extracted therein.

As used herein, the term “solvent recovery” refers to a process of renewing used solvent to an extent sufficient for its use again as an extraction solvent. By no means does recovery require obtaining analytically pure solvent. In various embodiments, used solvent, even when “recovered” so that it may be used again, might still contain some impurities, e.g., volatile impurities that would otherwise be difficult or not cost effective to remove. Further, it may be that not all the solvent (e.g., by weight or volume) can be recovered. For example, if 1 liter of ethanol is used to extract a certain amount of oil from a plant, the solvent recovered in accordance with the present disclosure may measure to less than 1 liter. Also, in this 1 liter ethanol example, the recovered ethanol may not be 100% analytically pure ethanol, or may otherwise be less pure than the ethanol used in the first extraction. In various embodiments, solvent recovery yields about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.9%, 99.99% or 99.999% by weight or volume, based on the starting weight or volume of the solvent subjected to the recovery process. In various embodiments, the recovered extraction solvent is least 50% pure, or about 55%, 60%, 65%, 70%, 75%, 80%, 85%, 90%, 95%, 96%, 97%, 98%, 99%, 99.9%, 99.99% or 99.999% pure.

As used herein, the term “liquid extraction mixture” refers to the material subjected to the system, method and apparatus of the present disclosure. By no means does the term “extraction” limit the scope of “liquid extraction mixture.” Broadly defined, “liquid extraction mixture” includes any essentially non-volatile substance or non-volatile solute dissolved in a solvent, wherein the mixture can be subjected to fractionation to separate solute and solvent. In various embodiments, a liquid extraction mixture comprises one or more extraction solvents (as defined above) along with various materials extracted from a natural source, such as terrestrial or aquatic plant material. The various materials, such as natural products, may be dissolved, dispersed or solubilized in the extraction solvent(s). In various embodiments, a liquid extract mixture may be clear, cloudy, more than one phase, colorless or colored. Cloudiness, and/or presence of precipitates, may indicate certain natural products are not entirely dissolved, but may instead be solubilized to some degree and/or dispersed by the extraction solvent. In various embodiments, a liquid extraction mixture comprises an extraction solvent, e.g., ethanol, and a variety of polysaccharides and proteins extracted from algal material. In various embodiments, a liquid extraction mixture comprises an extraction solvent, e.g., ethanol, and a variety of fatty acids, essential oils, alkaloids, or polysaccharides extracted from nut, fruit, plant, flower, tree or seed materials. In various embodiments, a liquid extraction mixture comprises an extraction solvent, e.g., ethanol, and a variety of cannabinoids extracted from plant material. In various embodiments, a liquid extraction mixture may comprise from about 1% by weight to at least about 50% by weight cannabinoids (i.e., 1:1 loading), based on the total weight of the mixture. In various embodiments, the liquid extraction mixture may comprise from about 1% by weight to about 30% by weight cannabinoids, based on the total weight of the mixture. In certain examples, a liquid extraction mixture may comprise 75% by weight ethanol and 25% by weight cannabinoid materials. In other examples, a liquid extraction mixture may comprise 90% by weight ethanol and 10% by weight cannabinoid materials. In still other examples, a liquid extraction mixture may comprise 99% by weight ethanol and 1% by weight cannabinoid materials. A liquid extraction mixture for use in the system, method and apparatus of the present disclosure may be pressurized and heated, such as by placing the mixture in a liquid feed tank that can be pressurized and that has a heating jacket or submersion heater to heat the liquid. In other embodiments, the liquid extraction mixture is heated as it approaches the point of atomization, resulting in less wasted heat and shorter time at elevated temperatures for temperature sensitive substances.

As used herein, the term “fluid” or “fluidic” refers broadly to liquids, gases, and aerosols. In various embodiments, two components of an apparatus may be in “fluidic communication,” and the material that moves from one component of the apparatus to another component in the apparatus may comprise a liquid, gas or an atomized fog.

As used herein, the term “engine oil” refers broadly to those heavy fluids typically referred to as motor oil, pump oil, or lubricating oil. Thus, for the sake of simplicity, “engine oil” is used universally throughout the present disclosure to mean all of these different types of industrial machine oil. Typically, these fluids are C18-C34 hydrocarbon based, derived from crude oil, but also containing other components based on the intended use for the fluid. For example, some engine oils used in internal combustion engines may purposely contain octane, detergents, antiknock agents, anticorrosive agents, etc. Used engine oil, on the other hand, may contain all sorts of unintentionally entrained contaminants from use, including solvents and metals. Used vacuum pump oil will likely contain dissolved solvents and other volatiles and essentially non-volatiles (higher molecular weight organic substances like fatty acids, terpenes, triterpenes, etc.), especially if a cold trap positioned just before the vacuum pump was not properly maintained at a cold temperature (e.g., with dry ice) during use of the vacuum pump, and especially in those instances where such as a pump was connected to a vacuum manifold or other structure where solvents and other organics were present. In various embodiments, used engine oil may be fractionated in accordance with the present disclosure, and the recovered oil and the dissolved solvents and other materials recycled and reused.

As used herein, the term “salt water” refers to any ionic strength aqueous solution comprising a dissolved salt. In various embodiments, salt water herein is river, stream, sea or ocean water, alternatively referred to collectively as “sea water.” In other embodiments, salt water comprises ordinary potable water, such as produced by a municipality or obtained from a well or other aqueduct system, comprising various dissolved hard water minerals such as calcium. In other embodiments, salt water is heavily laden with minerals and salt as is found in produced fluids during oil and gas production as well as the water used for injection for various purposes including acidification, fracturing, or other down hole processes. This waste-water requires treatment and can be quite brackish. In the methods disclosed herein, “salt water” is “desalinated” to produce distilled water or drinking water that is substantially distilled water. In various embodiments of the methods herein, the “sea salt” or “solar salt” is also recovered from the desalination process. In other methods, water is treated so that is ready for re-injection or discharge while the contaminants are concentrated for subsequent treatment or disposal. In the methods disclosed herein, well water or other ground water is purified to produce distilled water or drinking water that is substantially distilled water, and the minerals containing calcium and/or potassium discarded or repurposed into fertilizer.

As used herein, the term “cannabinoid” (or alternatively, “cannabinoid oil”) broadly refers to those compounds having cannabinoid receptor type-1 or receptor type-2 activity, either found naturally occurring in various Cannabis plant species or other terrestrial plants, or obtained by synthetic organic transformations starting from naturally occurring substances. Cannabinoids of interest herein include, but are not limited to, tetrahydrocannabinol (THC), tetrahydrocannabinolic acid (THC-acid), cannabidiol (CBD), cannabidiolic acid (CBD-acid), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE) and cannabicitran (CBT). As evident from this list, some of the cannabinoids of interest herein are characterizable as carboxylic acids (e.g., THC-acid), and in various embodiments of the present disclosure, it may be desirable to convert a particular cannabinoid carboxylic acid into its corresponding decarboxylated cannabinoid. For purposes herein, the cannabinoids are referred to as cannabinoid oils, and are thought of as essentially non-volatile substances.

As used herein, the term “decarboxylation” refers to a reaction in organic chemistry whereby a carboxyl group is removed from a starting molecule, thus liberating CO₂. The reaction may be generalized by the reaction equation: R—CO₂H→R—H+CO₂, often referred to as hydro-decarboxylation because of the resulting hydrocarbon R—H. A well-known decarboxylation that fits this equation, which is of interest herein, is the decarboxylation of Δ9-tetrahydrocannabinolic acid (THC-acid) to Δ9-tetrahydrocannabinol (THC).

### Single Tank Burst Atomization Fractionation System

In various embodiments, a basic system in accordance with the present disclosure comprises a single tank burst atomization and reflux apparatus. A single tank apparatus may be used for at least any of the three following purposes: (1) fractionation of a mixture of volatiles; (2) fractionation of a mixture of volatiles and essentially non-volatiles; and (3) fractionation of a mixture of volatiles and non-volatile solutes. In various embodiments, mixtures for fractionation may comprise any combination of volatiles, essentially non-volatiles, and non-volatile solutes.

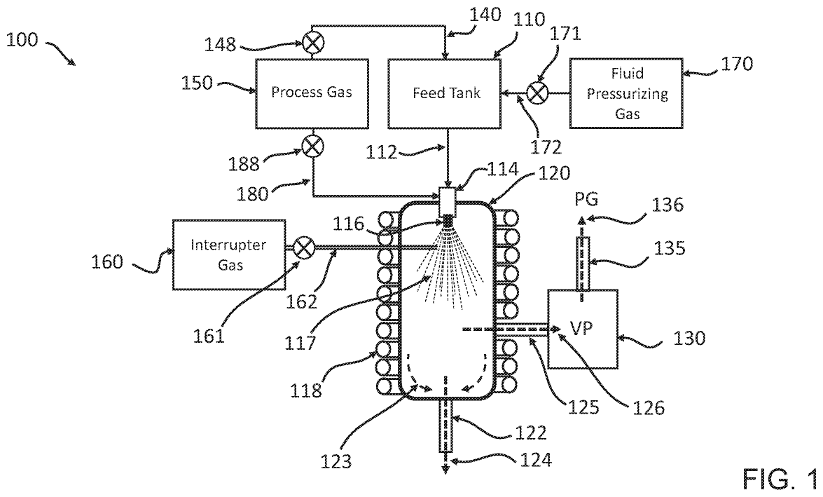

FIG. 1 illustrates a basic single-tank burst atomization fractionation apparatus 100 in accordance with various embodiments of the present disclosure. With reference to FIG. 1, fractionation apparatus 100 comprises a number of components fluidically connected to each other such that fluid flow can occur between components. Pipe diameters, fittings, pressure regulators and valve placements may be varied as necessary, and the relative sizes of these and other elements, or their presence or absence, should not be taken as literal or limiting in any way when viewing FIG. 1. For example, additional valves and pressure regulators may be configured onto various fluid transfer lines as needed. Most importantly, various components may not be illustrated in this schematic, or optional components may be illustrated. In the various drawing figures, a heavy dashed line with a directional arrow indicates a fluid flowing in the illustrated direction, such as through a pipe. In some instances, and to simplify drawings, a single black line, rather than parallel lines, may be used to illustrate a fluidic transfer line.

As illustrated generally in FIG. 1, fractionation apparatus 100 comprises a feed tank 110 to contain a liquid mixture to be fractionated, an atomization chamber 120, and a vacuum pump 130. As shown in the schematic, these components are connected in series and in fluidic communication with one another such that fluid can move through the apparatus as indicated by the dashed arrows. The use of the term “conduit” or “transfer line” does not limit the configuration of these passageways and fittings in any way. Components may be connected directly to one another, such as abutting, without use of any measurable length of piping. Transfer lines may be as narrow or as wide in diameter as necessary. Components may be equipped with fittings so that a system can be assembled, disassembled and rearranged in a modular sense. The atomization chamber 120, may be constructed from stainless steel, aluminum, cast iron, glass, or other suitable material. Each of these components may vary in design complexity, such as comprising additional internal elements. In various embodiments, an atomization chamber may comprise a heat exchanger, internal baffles, etc. as necessary.

With continued reference to FIG. 1, heating and/or cooling coils 118 or a suitable heating/cooling jacket, or combinations thereof, are provided around the outside and/or the inside walls of the atomization chamber 120. Such a design configuration should not be assumed to provide only a fixed temperature in the atomization chamber 120. In various embodiments, the temperatures of the interior walls and interior space of the atomization chamber 120 may be regulated as needed, and can, in various embodiments, be programmed to ramp up and/or ramp down in a process designed to invoke certain vapor pressures within the chamber 120 and to induce fractionation between multiple components of a mixture. For example, the heating/cooling coils 118 or jackets may be dual use—heating and cooling, and portions of the chamber 120 can be heated and/or cooled, and can be ramped up or down in temperature in particular zones in and around the chamber 120 to achieve the desired fractionations. For example, the top part of the chamber 120, such as near the location of atomization may be heated, whereas the lower portion of the chamber 120 may be cooled, and the lower portion may be ramped down in temperature to change the composition of the material condensed.

With continuing reference to FIG. 1, fluid transfer through the fractionation apparatus 100 is the result of pressuring at the front end, such as with the pressurized process gas tank 150 and/or the fluid pressurizing gas tank 170, and/or pulling a vacuum at the back end, such as with the vacuum pump (VP) 130. For example, a liquid mixture requiring fractionation, contained within the feed tank 110, is pressurized by the pressurized tank 150, containing a process gas, connected to the liquid feed tank 110 via the gas line 140 and pressure regulating valve 148 so that the now pressurized mixture can be directed through line 112 to the atomization fitting 114. In various embodiments, a fluid pressurizing gas is provided in fluid pressurizing gas tank 170, which is connected to the liquid feed tank 110 via the transfer line 172, regulated by the pressure regulator 171. In various embodiments, the process gas is the same as the fluid pressurizing gas. In other embodiments, these gasses are different, depending on the liquid mixture to be fractionated. As shown in FIG. 1, the process gas may also be directed into the atomization nozzle fitting 114 through transfer line 180, regulated by pressure regulator 188 so as to merge with the pressurized fluid to be fractionated in the head of the atomizing nozzle and to maintain turbulent flow in the atomization chamber. Transfer line 112 between pressurized liquid feed tank 110 and the atomization nozzle fitting 114 may be jacketed or fitted with gas burners such that the line 112 can be heated. Optionally, or additionally, the atomization nozzle fitting 114 may be jacketed and heated. By the combination of process gas tank 150, and the heating of the transfer line 112 and/or heating of the atomization nozzle fitting 114, the mixture at the inlet to the atomization nozzle fitting will be at an initial temperature and an initial pressure indicated herein as “P1/T1.” That is, P1 indicates the initial pressure of the mixture to be fractionated at the inlet to the atomization nozzle fitting 114 and T1 indicates the initial temperature of the mixture to be fractionated at the inlet to the atomization nozzle fitting 114. As discussed below, this heated and pressurized mixture is atomized into a fog across a pressure and temperature gradient.

With further reference to the fractionation apparatus 100 in FIG. 1, the pressurized and heated mixture entering the atomization nozzle fitting 114 from line 112 is atomized into the atomization chamber 120 by expulsion through an atomization nozzle 116. The details of the atomization nozzle are discussed below in reference to the other drawing figures. In the schematic of FIG. 1, the nozzle 116 can be either an atomizing nozzle or an ultrasonic nozzle, recognizing that the feed liquid to an ultrasonic nozzle need not be pressurized. In various embodiments, there may be more than one inlet into the atomization chamber 120, such as a plurality of atomization nozzle fittings and atomization nozzles and/or ultrasonic nozzles (i.e., multiples of the 114/116 combination) operating in parallel to increase throughput.

With continued reference to FIG. 1, the liquid mixture requiring fractionation is atomized into droplets 117 as shown inside the atomization chamber 120. A disrupter gas 160, discussed below, may be used to affect the atomized droplet size, and this gas may be deployed by injection through valve 161 and injection line 162, directly into the atomization fog as illustrated. In various embodiments, the injector gas and the process gas may be the same gas, and thus only a single tank is needed. The interior of the chamber 120 is maintained at a pressure P2 that is less than the inlet pressure P1 of the pressurized liquid mixture. In this way, the liquid is atomized across a pressure drop since P1>P2. The pressure difference, P1-P2, may be slight up to huge. The pressure/vacuum P2 inside the atomization chamber 120 may be regulated by the vacuum pump 130 in fluidic communication with the atomization chamber 120. Thus, in some ways the vacuum pump 130 may be seen as pulling mixture through the apparatus and controlling the pressure P2 in the atomization chamber 120. As noted above, additional pressure valves and regulators can be added where necessary, such as along transfer line 112 leading into the atomization chamber, and/or in transfer line 125 connecting vacuum pump 130 to the atomization chamber 120.

In various embodiments, P2 may be greater than, equal to, or less than atmospheric pressure (760 mmHg). The atomization chamber 120 is at a temperature T2, wherein in various embodiments, T1=T2, or in other embodiments, T1>T2, or T2>T1. In various embodiments, T2 is not held constant but instead follows a programed cycle where T2 may be ramped up or down, or toggled up and down such as in cycles, as needed for a particular fractionation process. As mentioned, various portions of the interior walls of the chamber 120 may be at different temperatures at any given time in the process. Thus, moving along an interior wall of the chamber 120 from a location near the atomization nozzle 116 to another location near a liquid outlet 122, different temperatures T1′, T1″, T1′″, and so forth may be provided, and none of these localized temperatures need to remain constant. Thus, T1′, T1″, T1′″ etc. may follow a programed cycle. The temperature programming on the interior wall of the atomization chamber 120 provides fractionation.

With further reference to FIG. 1, the atomized mixture 117, in the form of an atomic fog or mist, fractionates to some degree by impingement and coalescence of the atomized droplets on the interior wall of the chamber 120. This condensation on the wall of the chamber 120 is indicated in FIG. 1 as fluid flow 123. As T2 is varied, the composition of 123 changes.

The most volatile components of the beginning mixture in liquid feed tank 110 may exit as a vapor 136, mixed with the process gas (PG) through the vacuum pump exit 135. These vapors may be collected and condensed after exiting the pump through outlet 135, or they may be trapped in a cold trap positioned along transfer line 125 between atomization chamber 120 and vacuum pump 130. Thus, in the presence of a cold trap, such as a trap filled with dry ice, vapor 126 can be condensed and collected short of the vacuum pump 130. Various reasons to trap and condense vapor 126 include avoiding dissolution of the vapor 126 into the engine oil present in the vacuum pump 130. Regardless, used engine oil can also be fractionated in various embodiments of the fractionation apparatus 100, as discussed below.

In various embodiments, the process gas (PG), or pressurized gas, may comprise any inert gas such as CO₂, N₂ or Ar. In some examples, the process gas may comprise pressurized air.

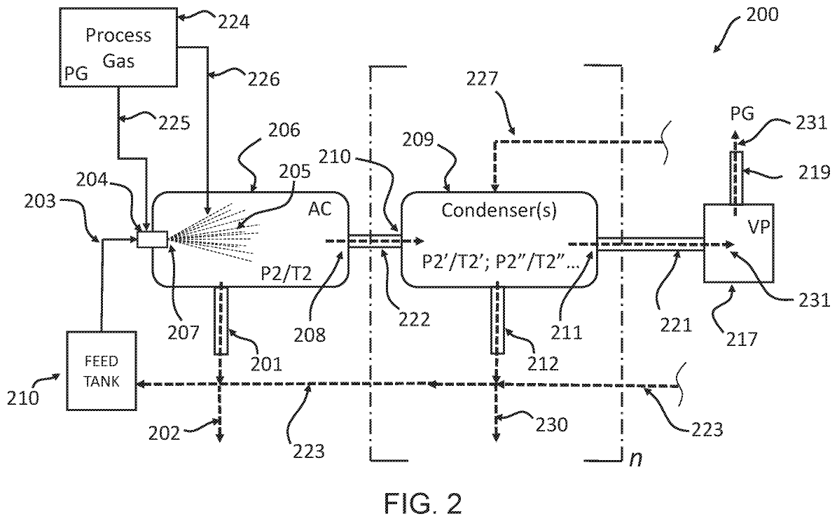

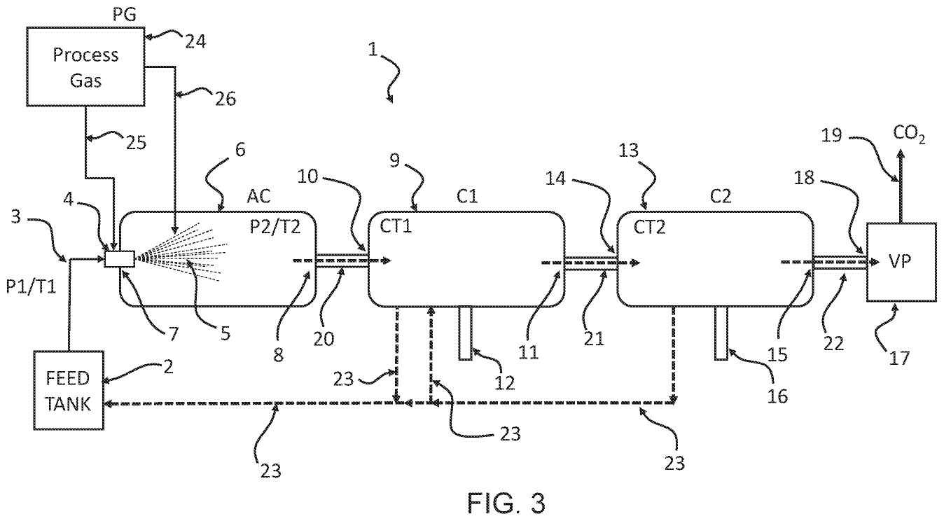

Fractionation apparatus 100 may further comprise any number of recirculation pathways (not illustrated in FIG. 1, but are discussed in reference to FIGS. 2 and 3). For example, vapors and/or aerosols 126 from the atomization chamber 120 may be recycled back into the liquid feed tank 110 or directly injected into transfer line 112 or the atomization nozzle fitting 114, in order to atomize the recycled fraction. In this way, recycling acts in a similar way to continuous liquid-liquid extraction. Recirculation pathways may be implemented when the liquid 124 from outlet 122 and/or the vapors 126/136 from outlet 125/135 do not meet predetermined standards, such as purity. In various embodiments, controlled and efficient production of high surface area atomized particles 117 provides efficient fractionation of a complex liquid extraction mixture, and these recirculation pathways can be implemented as needed to optimize this fractionation process.

With continued reference to FIG. 1, various embodiments of a fractionation apparatus 100 may further comprise a disrupter gas. A single tank 150/160 may contain and dispense into two locations a single gas that provides dual function as both a process gas and a disruptor gas. The disrupter gas may be fed directly into the atomization nozzle fitting 114 to merge with the heated and pressurized fractionation mixture, and/or injected into the atomization chamber through valve 161 and injection line 162 and directly into the mist of atomized droplets 117, in order to affect the particle size of the droplets. It's important to note that a single gas, e.g., CO₂, pressurized in process gas tank 150, may function as both a feed gas to pressurize and propel the liquid mixture in tank 110 through the atomization nozzle 116, and a disruptor gas to affect the atomized droplets.

In various embodiments, the single tank burst atomization fractionation apparatus 100 depicted in FIG. 1, with the necessary temperature and pressure programming, optional valve placement and recirculation lines, can be used to fractionate mixtures comprising any combination of volatiles, essentially non-volatiles, and non-volatile solutes.

Recovery of Volatile Solvents from a Mixture of Same

As mentioned, the burst atomization fractionation system, for example, as illustrated in FIG. 1, can be used to separate mixtures of volatile substances. For example, a number of industrial processes result in mixtures of volatile solvents that require separation prior to reuse. Such processes include, for example, natural rubber (polyisoprene) extraction from tree or plant material or resin, and recrystallization procedures in bulk pharmaceutical operations used to purify drug actives and precursors. In these and other processes, mixtures may comprise, for example, pentanes, hexanes, cyclohexane, acetone, petroleum ethers, benzene, toluene, xylenes, chloroform, methylene chloride, ethyl acetate, and other volatile solvents. The mixture obtained from such a process can be separated into its components by using the apparatus of FIGS. 1-3. The main variables to control include P1 and T1, and the gradations of P2 and T2, particularly the gradation of T2 along portions of the interior wall of the atomization chamber 120, and a programmed increase or decrease in P2 in the chamber. As these variables are ramped up or down, or in the case of T2, moved up or down along regions of the interior wall of the chamber 120, the composition of the liquid 124 emerging from outlet 122 changes accordingly, and this liquid is collected into different vessels as the composition of 124 changes. This collection is much like fractional distillation where a rotating collar at the end of the condenser can accommodate several flasks and the collar is rotated as the fractions coming from the condenser changes. In this case, the walls of the atomization chamber function as the condenser in fractional distillation, and the atomization droplet size, dispersion, and direction (such as to purposely impinge on the walls) act as the fractionating column in fractional distillation. Thus, in a simple example, ramping down T2 will sequentially provide methylene chloride first from outlet 122 followed by ethyl acetate from outlet 122, assuming a starting mixture of methylene chloride and ethyl acetate.

Recovery of Volatile and Essentially Non-Volatile Materials from a Mixture of Same

As mentioned, various embodiments of a burst atomization fractionation apparatus, including the embodiments encompassed in the generalized illustration of FIG. 1, can be used to separate volatiles from essentially non-volatile materials. One specialized example of this is separation of ethanol from cannabinoid oils, which is discussed in more detail below in reference to FIG. 3. Another example, used for proof of concept herein, is separating olive oil and ethanol. In general, the apparatus depicted in FIGS. 1-3 can be used to separate essentially non-volatile oils and volatile solvents. In various embodiments, oil is collected as liquid 124 emerging from outlet 122, and the volatile solvent(s), such as ethanol, collected as vapor 126 from the vacuum outlet 125, preferably condensed into a liquid within a suitably chilled trap positioned inline between the atomization chamber 120 and the vacuum pump 130. Depending on the complexity of the beginning mixture of essentially non-volatile oil and volatile solvent(s), P1/T1 and P2/T2 can be set and/or varied as needed to affect separation. In various embodiments, P1/T1 and P2/T2 may be programmed to change incrementally, such as in gradations. In other embodiments, it may be desirable to change T2 along portions of the interior wall of the atomization chamber 120 to promote effective separation of the oil components and the volatile solvent components in a complex mixture. Depending on how complicated the oil and solvent mixture is, one or more condensers can be added to the system, along with optional recirculation lines, as explained in the example of cannabinoid purification and ethanol recovery (FIG. 3).

### Recycling of Used Engine Oil

Recovery and reuse of used engine oil is highly desired, at least for environmental reasons. Used engine oil has a long list of contaminants, including dissolved gasoline constituents like ethanol, hexane and octane if the engine was a gasoline combustion engine, cleaning additives, anticorrosive agents, cooling agents, pour point depressants, and a host of metals such as iron, nickel, copper, lead and zinc. Although extraction of contaminants from used engine oil using a composite of solvents is somewhat effective, the cost is prohibitive. Extraction of contaminants using strong acids is financially cheaper, but the method is primarily effective at removing only the metal contaminants.

Following the examples above, and depending on the compositional complexity of the used engine oil, P1 and T1, and the gradations of P2 and T2, particularly the localities in variations of T2 along portions of the interior wall of the atomization chamber 120, and a programmed increase or decrease in P2 in the chamber 120, are the variables used to promote effective separation of the components in a complex mixture, such as used engine oil containing both volatiles like hexane, octane and ethanol and non-volatile solutes like lead and zinc. Depending on how complicated the composition of the used engine oil is, one or more condensers can be added to the system, along with optional recirculation lines, as explained in the multi-condenser variations described below.

### Desalination of Sea Water and Purification of Drinking Water

In various embodiments, the apparatus 100 depicted in FIG. 1 is adaptable for energy efficient desalination of sea water. In this case, baffles or removable trays can be disposed within atomization chamber 120 for salt collection. For example, a metal sheet positioned orthogonal to the spray direction can be heated, whereupon atomized spray 117 comprising sea salt and water impinges upon the sheet, with the water vaporizing and condensing as liquid 123 along the walls of the chamber 120. In this way, salt is collected on the removable baffles, trays or sheets, whereas the water exits as liquid 124 from the outlet 122. In various embodiments, the jacket or coils 118 are cooled, such that all or almost all of the water in the sea water is condensed and collected as liquid 124 rather than exiting the atomization chamber 120 as steam 126. In various embodiments, the saltwater, previously filtered to remove particulates, is fed to the atomization chamber where a very brackish super-concentrated saltwater is collected to discharge for separate drying and recovery of sea salt. The purified water is collected through condensation elsewhere. In various embodiments, the liquid fed to the atomization nozzle can be pressurized simply via head pressure, by configuring a height to the fluid column.

In drinking water purification, such as to purify well water, the same apparatus is used, wherein the purified water 124 exiting from the outlet 122 may be pumped up to usable pressure, such as household pressure to power various faucets and showers in a residence. The purified water 124 can substitute for RO water in institutions and households.

### Multi-Tank Burst Atomization Fraction System

In various embodiments, one or more condensers may be necessary in the apparatus, disposed between the atomization chamber at the beginning of the system and the vacuum at the end of the system. Such a variation is illustrated by the general schematic in FIG. 2. “Multi-tank” herein refers to one or more condensers rather than additional atomization chambers. Recall that throughput can be improved simply by equipping one atomization chamber with multiple atomization nozzles and to configure the apparatus with the appropriate number of additional feed lines from liquid feed tanks. Output in this case is improved, both in quality and quantity, by the addition of condensers.

In various embodiments, a multi-tank burst atomization apparatus such as illustrated in FIG. 2 is capable of fractionating any combination of volatiles, essentially non-volatiles and non-volatile solutes in mixtures, in a similar way as the single tank apparatus in FIG. 1.

With reference now to FIG. 2, a multi-tank burst atomization fractionation apparatus 200 is illustrated. The integer n indicates the number of condensers 209 in the system. When n=0, the system is substantially similar to the single-tank atomization fractionation apparatus 100 depicted in FIG. 1, i.e., wherein the interior walls of the atomization chamber function as the condenser, and various aspects of the atomization chamber may be associated with T2/P2, ramping up or down as necessary for the particular mixture. In the apparatus of FIG. 2, n can be 1 to about 10. In various embodiments, the condensers 209, 209′, 209,″ and so forth, in a multi-condenser configuration, need not be identical in materials of construction, physical dimensions and internal structures, or in the parameters set for each condenser. For example, the condensers may be adjusted to P2′/T2′ for 209, P2″/T2″ for 209′, P2′″/T2′″ for 209″, etc., depending on the number of condensers. Further, each condenser may include unique internal features such as baffles.

In FIG. 2, multi-tank fractionation apparatus 200 comprises a liquid feed tank 210, an atomization chamber 206, and a vacuum pump 217. Heating and/or cooling coils or jackets on the atomization chamber 206 are not illustrated (see FIG. 1 for an example). A liquid mixture requiring fractionation is contained within liquid feed tank 210, and is pressurized by the pressurized tank 224 at the atomization fitting 204. This configuration, in contrast to FIG. 1 where the liquid feed tank (110) is pressurized, makes it simpler to bring materials back into the liquid feed tank 210 from various recirculation lines. Transfer line 203 may be jacketed or fitted with gas burners such that the line 203 is heated, and/or the atomization nozzle fitting 204 may be jacketed and heated. The mixture at the inlet to the atomization nozzle fitting 204 will be at an initial temperature and an initial pressure indicated herein as “P1/T1.” That is, P1 indicates the initial pressure of the mixture at the inlet to the atomization nozzle fitting 204 and T1 indicates the initial temperature of the mixture at the inlet to the atomization nozzle fitting 204. This heated and pressurized mixture is atomized by the atomizer 207 into a fog 205 across a pressure and temperature gradient.

In the apparatus 200 of FIG. 2, the atomizer 207 can be either an atomizing nozzle or an ultrasonic nozzle, recognizing that the feed liquid to an ultrasonic nozzle need not be pressurized, in which case the transfer line 203 need only be pressurized to a sufficient pressure to move the mixture from the liquid feed tank 210 to the atomization nozzle fitting 204. In various embodiments, there may be more than one inlet into the atomization chamber 206, such as a plurality of atomization nozzles and/or ultrasonic nozzles operating in parallel to increase throughput by simply atomizing more material into the atomization chamber 206.

In various embodiments, the inlet pressure P1 of the liquid mixture feed through the atomization nozzle fitting 204 and into the atomization nozzle 207 may be from just greater than about atmospheric pressure (1.02 bar, 765 mmHg) to up to at least about 100 bar (75,000 mmHg). The inlet temperature of the mixture T1 may be from just about ambient 23° C. to at least about 500° C. These exemplary P1 and T1 ranges are not intended to be limiting in any way. Other initial temperatures and/or pressures outside these ranges may be found optimal for particular fractionation processes and/or to promote desired chemical reactions in the fractionation process. As mentioned, the pressurized feed of the mixture to be fractionated may be split into any number of parallel lines leading to a plurality of atomization nozzles in order to accommodate higher throughput volumes.

The liquid extraction mixture is atomized into droplets 205 as shown inside the atomization chamber 206. A disrupter gas, as discussed above in reference to FIG. 1, may be used to affect the atomized droplet size. The chamber 206 is maintained at a pressure P2 that is less than the inlet pressure P1 of the pressurized inlet mixture. In this way, the liquid is atomized across a pressure drop since P1>P2. The pressure difference, P1-P2, may be slight up to huge. The pressure/vacuum P2 inside the atomization chamber may be regulated by the vacuum pump 217 placed at the end of the apparatus. In various embodiments, P2 may be greater than, equal to, or less than atmospheric pressure (760 mmHg). The atomization chamber is also maintained at a temperature T2, wherein in various embodiments, T1=T2, or in other embodiments, T1>T2, or T2>T1. As discussed in the context of FIG. 1 and apparatus 100, T2 and P2 are not necessarily held constant, e.g., they may be programmed to ramp up or down, and portions of the interior wall of the atomization chamber 206 may be set at different temperatures and programmed at different cycles than other portions of the interior wall. This programming is relevant to the composition of the condensate 202, if present, that may be collected from outlet 201 disposed in the atomization chamber 206. As per apparatus 100 in FIG. 1, this output can be recycled back into the liquid feed tank 210 in a continuous liquid-liquid fractionation configuration, particularly if collected condensate 202 remains an unusable mixture requiring further processing.

The atomized mixture 205 that is not purposely condensed into liquid output 202 moves out outlet 208, through transfer line 222 and into the first condenser 209 (or into the only condenser in the embodiments where n=1). Condenser 209 is maintained at a temperature of T2′ and a pressure of P2′, in some instances wherein T2≠T2′ and/or P2 P2′. The term “movement” is a generalization of various processes, such as mist being pulled through the system via the vacuum pump 217, or other physical effects. Condensate 230 from the first or only condenser 209 may be collected from outlet 212. The material not otherwise condensed, i.e. vapor/aerosol 211 moves further into the next condenser, if n>1 and there are additional condensers, or through the outlet 221 to the vacuum pump 217 (or into an intermediate cold trap) as explained in reference to FIG. 1. Additional condensers beyond first condenser 209, if present, need not be held at the same temperature T2′. Additional condensers can be maintained at their own temperatures T2′, T2″, T2″, etc., which may be constant or variable, depending on the nature of the mixture undergoing fractionation and the number of condensers connected in series. Remaining vapors/aerosol 231 continue on through the vacuum pump 217 to be expelled into the environment at outlet 219, or collected in the appropriately configured and chilled cold trap positioned inline just before the vacuum pump 217. This final output 231 may consist mostly of the process gas when a cold trap is suitably maintained to collect any carryover vapors in the process gas. In some embodiments, a process gas such as CO₂ may be compressed and recycled.

With continued reference to FIG. 2, the apparatus 200 may further comprise any number of recirculation pathways 223 and 227, such as depending on the number of condensers and the complexity of the mixture in need of fractionation. For example, vapors/aerosols condensed from the atomization chamber 206 and/or from condenser 209 may be recycled back into the liquid feed tank 210 via a common line 223. Further, vapors/aerosols condensed from other condensers, when more than one condenser is present, may be recycled into the condenser immediately preceding in the series via recirculation line 227, or all the way back to the liquid feed tank 210 (return line not illustrated). Generally, recirculation pathways such as 223 may be implemented when the output 202 from outlet 201, the output 230 from outlet 212, and/or the output 231 from outlet 219 (or, products condensed any additional condensers) do not meet predetermined standards, such as purity. Controlled and efficient production of high surface area atomized particles 205 provides efficient fractionation of a complex liquid mixture, and these recirculation pathways 223 can be implemented as needed to optimize this fractionation process.

With continued reference to FIG. 2, an apparatus 200 may further comprise a process gas PG tank 224 to contain a pressurized process gas. A process gas may comprise a feed gas (pressurized so that the contents of the liquid feed tank 210 can be pressurized at the atomization nozzle fitting 204), or a disrupter gas. A single tank 224 may contain and dispense, into two locations, a single gas that provides dual function as both a feed gas and a disruptor gas. Depending on the function of the process gas, the gas may be fed through line 225 directly into the atomization nozzle fitting 204 to merge with the liquid mixture from transfer line 203, and/or through transfer line 226 into the atomization chamber 206 and directly into the mist of atomized droplets 205, in order to affect the particle size of the droplets. It's important to note that a single gas, e.g., CO₂, pressurized in process gas tank 224, may function as both a feed gas to pressurize and propel the liquid mixture through the atomization nozzle 207, and as a disruptor gas through line 226 to affect the atomized droplets.

In various embodiments, a fractionation apparatus 200 may further comprise any number of heat exchangers that function to recycle as least some of the energy released in the atomization process. For example, the large amount of energy put into heating and pressurizing a liquid extraction mixture prior to atomization is partly recoverable in the form of heat of condensation. With the appropriately configured heat exchangers, the heat of condensation may be recycled back to the heating of the liquid mixture prior to atomization, and/or the heating of the atomization nozzle fitting. Other heat exchanger configurations can be envisioned, so as not to waste energy supplied to the apparatus in the form of electricity, flame or steam. For example, the heat emanating from the vacuum pump 217 may be captured and recycled into upstream heating processes.

### Examples

A Multi-Tank Burst Atomization Fractionation Apparatus with Two Condensers

In various embodiments, a multi-tank burst atomization apparatus 1, comprising an atomization chamber 6 and two separate in-line condensers C1 and C2, as illustrated for example in FIG. 3, can be used to separate any combination of volatiles, essentially non-volatiles, and non-volatile solutes. Non-limiting examples of a multi-tank fractionation include separation of volatile solvent mixtures, separation of essentially non-volatile oils from volatiles, recovery of engine oil by separation of essentially non-volatile hydrocarbon from dissolved solvents and metals, and desalination of sea water and purification of drinking water.

In various embodiments, a mixture comprising volatile solvent, e.g., a lower alcohol extraction solvent, and natural products, e.g. cannabinoid oils, is fractionated through a multi-tank apparatus comprising two in-line condensers, such as the apparatus 1 in FIG. 3. In various embodiments, certain chemical reactions, e.g., decarboxylation, are desired, and these reactions can be accomplished through the fractionated process. In other words, an atomization fractionation apparatus in accordance with various embodiments of the present disclosure operates to fractionate mixtures and initiate desired organic reactions for one or more components in the mixture.

As a non-limiting example, a system, method and apparatus is described that provides both recovery of an extraction solvent and chemical reactions of the solutes present in the liquid extraction mixture thus fractionated. The present disclosure is not limited to the particular examples discussed below, and any variation of the burst atomization apparatus 100 in FIG. 1 (no separate condenser), apparatus 200 in FIG. 2 (where n=1 to 10), or the 2-condensor apparatus 1 in FIG. 3, may be employed for the particular fractionation. For example, the apparatus exemplified below and discussed in reference to FIG. 3 may be used to separate various essentially non-volatile natural products from a volatile extraction solvent used to extract the natural products from their natural source. For example, mixtures of vegetable oil and extraction solvent may be successfully fractionated into commercially useful vegetable oil and reusable solvent. Or, mixtures of essential oils, e.g., limonene, and extraction solvent may be successfully fractionated into commercially useful orange oil and reusable solvent. In general, a liquid extraction mixture comprising any natural product extracted from a nut, fruit, plant, flower, tree, root or seed material along with the extraction solvent, may be separated in the apparatus exemplified herein.

As a specific example, financially viable production of commercially useful cannabinoids (e.g., tetrahydrocannabinol (THC) and cannabidiol (CBD)) often requires a complex series of processing steps. These individual steps tend not to be combinable for at least the reason that the processing steps comprise both physical and chemical transformations. For example, a financially viable cannabinoid process typically includes both a way to recover/recycle the extraction solvent used to extract the naturally occurring cannabinoids from plant material, and a way to chemically transform the naturally occurring cannabinoids into useful oils, such as by decarboxylation reactions. Massive amounts of solvent (e.g., ethanol, isopropanol, ethers, petroleum, CO₂ and butane) are often used to extract naturally occurring cannabinoids from Cannabis plant material, and disposal is neither practical nor prudent. Further, several of the naturally occurring cannabinoids that are extracted from the plant are in the form of carboxylic acids that must be decarboxylated in order to obtain the commercially useful cannabinoids. Most notably, THC-acid and CBD-acid must be decarboxylated to THC and CBD, respectively.

Separate decarboxylation of cannabinoid carboxylic acids, such as to form commercially useful cannabinoids, is also problematic. Decarboxylation is usually accomplished by a short path system or falling film, both processes dependent upon heating large volumes or large surface areas of cannabinoid acids, which are energy inefficient processes. The thickness of the cannabinoid acid layer and the temperature must be controlled and manually observed because the quality of the precursor cannabinoid acid material varies greatly, requiring such systems to be adjusted accordingly. For example, in short path distillation systems, cannabinoid oils may foam or outgas, expanding uncontrollably and filling the entire system. Also, falling film processes require large surface areas and heating capacities to keep the film surfaces hot.

In various embodiments, atomization fractionation systems according to the present disclosure can be used to separate cannabinoids from an extraction solvent such as ethanol and effect concomitant decarboxylation of the cannabinoid acids into the commercially useful cannabinoids such as CBD.

With reference now to FIG. 3, a decarboxylation and solvent recovery apparatus 1, in accordance with various embodiments of the present disclosure, comprises a number of components fluidically connected to each other to allow fluid flow between the components. Various optional components may not be illustrated in this schematic for the sake of clarity. As illustrated generally in FIG. 3, an apparatus 1 comprises a liquid feed tank 2, an atomization chamber 6, a first condenser 9, a second condenser 13, and a vacuum pump 17. As shown in the schematic, these components are connected in series and in fluidic communication with one another such that fluid can move through the apparatus as indicated by the dashed arrows. For example, a connecting conduit 20 fluidically connects the atomization chamber 6 with the first condenser 9; a connecting conduit 21 fluidically connects the first condenser 9 with the second condenser 13; and a connecting conduit 22 fluidically connects the second condenser 13 with the vacuum pump 17. The use of the term “conduit” does not limit the configuration of these connections in any way. Components may be coupled directly to one another, such as abutting, without use of any measurable length of piping. Components may be equipped with fittings so that a system can be assembled, disassembled and rearranged in a modular sense.

The atomization chamber 6, first condenser 9 and second condenser 13 may be constructed from stainless steel, aluminum, cast iron, glass, or other suitable material. Each of these components may vary in design complexity, such as comprising additional internal elements. In various embodiments, an atomization chamber may comprise a heat exchanger and one or more condensers may comprise internal baffles, cyclones, cooling/heating coils, etc.

The apparatus functions as indicated by the various arrows in FIG. 3, showing fluid transfer. Fluid transfer through the apparatus is the result of pressuring at the front end and/or pulling a vacuum at the back end. For example, a liquid extraction mixture, contained within liquid feed tank 2, is pressurized and heated and atomized into the atomization chamber 6 by expulsion through an atomizer 4. These elements are described in more detail below and were also described in detail above in reference to FIGS. 1 and 2. In the schematic of FIG. 3, the atomizer 4 can be either an atomizing nozzle or an ultrasonic nozzle, recognizing that the feed liquid to an ultrasonic nozzle need not be pressurized. In various embodiments, there may be more than one inlet into the atomization chamber 6, such as a plurality of atomization nozzles and/or ultrasonic nozzles operating in parallel to increase throughput.

The liquid extraction mixture is atomized into droplets 5 as shown inside the atomization chamber 6. A disrupter gas, discussed below, may be used to affect the atomized droplet size. The chamber is maintained at a pressure P2 that is less than the inlet pressure P1 of the pressurized liquid extraction mixture. In this way, the liquid is atomized across a pressure drop since P1>P2. The pressure difference, P1-P2, may be slight up to huge. The pressure/vacuum P2 inside the atomization chamber may be regulated by the vacuum pump 17 placed at the end of the apparatus. In various embodiments, P2 may be greater than, equal to, or less than atmospheric pressure (760 mmHg). The atomization chamber is also maintained at a temperature T2, wherein in various embodiments, T1=T2, or in other embodiments, T1>T2, or T2>T1. The atomized mixture 5 moves into the first condenser 9 maintained at a temperature of CT1. The term “movement” is a generalization of various processes, such as mist being pulled through the system via the vacuum pump 17, or other physical effects. Condensate from first condenser 9 may be collected at outlet 12. The material not otherwise condensed moves further into second condenser 13 maintained at temperature CT2. Condensate from second condenser 13 may be collected at outlet 16. Remaining vapors/aerosol continues on through the vacuum pump 17 and is expelled into the environment at outlet 19. This final output may consist mostly of CO₂, such as when a process gas comprises CO₂ and/or when concomitant chemical reactions occurring during atomization comprise decarboxylation. In some embodiments, CO₂ may be compressed and recycled as an input to the system such that CO₂ is used to “start” the system but then accumulates through recycle and outgassing from decarboxylation reactions.

With continued reference to FIG. 3, a decarboxylation and solvent recovery apparatus 1 may further comprise any number of recirculation pathways 23. For example, vapors/aerosols from first condenser 9 may be recycled back into the liquid feed tank 2, and/or vapors/aerosols from second condenser 13 may be recycled back into first condenser 9 or all the way back to the liquid feed tank 2. Recirculation pathways 23 are discussed in more detail below. Generally, the outlet 12 from the first condenser 9 provides a nonvolatile mixture of cannabinoids and/or other natural products, wherein the outlet 16 from the second condenser 13 provides recycled extraction solvent. Recirculation pathways may be implemented when the output from outlet 12 and/or the output from outlet 16 do not meet predetermined standards, such as purity. In effect, the decarboxylation and solvent recovery apparatus 1 operates as a fractionator. Controlled and efficient production of high surface area atomized particles 5 provides efficient fractionation of a complex liquid extraction mixture, and these recirculation pathways 23 can be implemented as needed to optimize this fractionation process.

With continued reference to FIG. 3, a decarboxylation and solvent recovery apparatus 1 may further comprise a process gas PG tank 24 containing a process gas. A process gas may comprise a feed gas (pressurized so that the contents of the liquid feed tank 2 can be pressurized), or a process gas may comprise a disrupter gas. A single tank 24 may contain and dispense into two locations a single gas that provides dual function as both a feed gas and a disruptor gas. Depending on the function of the process gas, the gas may be fed through line 25 directly into the atomization nozzle to merge with the liquid extraction mixture, and/or through line 26 into the atomization chamber and directly into the mist of atomized droplets, in order to affect the particle size of the droplets. It's important to note that a single gas, e.g., CO₂, pressurized in process gas tank 24, may function as both a feed gas to pressurize and propel the liquid extraction mixture through the atomization nozzle 4, and a disruptor gas to affect the atomized droplets.

In various embodiments, a decarboxylation and solvent recovery apparatus 1 may further comprise any number of heat exchangers that function to recycle as least some of the energy released in the atomization process. For example, the large amount of energy put into heating and pressurizing a liquid extraction mixture prior to atomization is partly recoverable in the form of heat of condensation. In various examples, condensation may be directly on a surface of a heat exchanger strategically positioned in the atomization chamber. Further, various chemical reactions, such as decarboxylation, may be exothermic, and some of that heat may be recovered. With the appropriately configured heat exchangers, the heat of condensation and/or the heat produced in various exothermic chemical reactions may be recycled back to the heating of the liquid extraction mixture prior to atomization, and/or the heating of the atomization nozzle. Other heat exchanger configurations can be envisioned, such as not to waste energy supplied to the apparatus in the form of electricity, flame or steam. For example, the heat emanating from the vacuum pump 17 may be captured and recycled into upstream heating processes.

### A. Pressurized and Heated Inlet Mixture:

In various embodiments, a decarboxylation and solvent recovery apparatus processes a liquid extraction mixture, i.e., fractionates it. Liquid extraction mixtures may be held in the liquid feed tank, which can be pressurized by a gas feed comprising compressed N₂, Argon, or other compressed gas held, for example, in process gas tank 24. The gas feed provides pressure control of the liquid feed tank or the liquid extraction mixture at the inlet to the nozzle 4 and, consequently, control of the feed rate through the nozzle and into the atomization chamber. A pressure regulator may be used to control the pressure from the pressurized gas feed to the liquid feed tank containing the liquid extraction mixture or to the nozzle 4. An optional pressure gauge may be employed to monitor the pressure of the pressurized liquid extraction mixture at the inlet side of the atomization chamber. Other configurations can be envisioned that provide pressurized and heated liquid extraction mixture to the inlet of the atomization chamber. For example, an atomization nozzle may provide for heating liquid mixture upon entry to the nozzle.

In various embodiments, the inlet pressure P1 of the liquid extraction mixture feed to the atomization nozzle may be from just greater than about atmospheric pressure (1.02 bar, 765 mmHg) to up to at least about 100 bar (75,000 mmHg). The inlet temperature of the mixture T1 may be from just about ambient 23° C. to at least about 500° C. These exemplary P1 and T1 ranges are not intended to be limiting in any way. Other initial temperatures and/or pressures outside these ranges may be found optimal for particular fractionation processes and/or chemical reactions. As mentioned, the pressurized feed of liquid extraction mixture may be split into any number of parallel lines leading to a plurality of atomization nozzles in order to accommodate higher throughput volumes.

### B. Atomization Chamber:

In various embodiments, and with continued reference to FIG. 3, the decarboxylation and solvent recovery apparatus 1 comprises an atomization chamber 6 having an inlet 7 and an outlet 8. The inlet 7 is fitted with an atomization nozzle 4 that provides the atomization 5 of the heated and pressurized liquid extraction mixture. The liquid extraction mixture is fed in by supply line 3 to the atomization chamber 6, and in various embodiments is merged with a pressurized feed gas supplied from tank 24. Optionally, the inlet 7 to the atomization chamber is fitted with an ultrasonic nozzle 4 (i.e., nebulizer), in which case the liquid feed from line 3 does not need to be pressurized, although the liquid feed may still be heated to T1. The inside of the atomization chamber is maintained at a pressure P2 that is at least somewhat less than P1. The pressure inside the atomization chamber may be controlled and maintained by the vacuum pump 17 fluidically connected downstream in the apparatus. In various embodiments, the pressure P2 inside the atomization chamber 6 may be from about 1 bar (750 mmHg) down to about 0.00013 bar (0.1 mmHg). The temperature T2 inside the atomization chamber 6 may be maintained at from about ambient temperature (23° C.) up to about T1 (maximum about 500° C.). These exemplary P2 and T2 ranges are not intended to be limiting in any way. Other atomization chamber temperatures and/or pressures P2 and T2 outside these ranges may be found optimal for particular fractionation processes and/or chemical reactions.

Atomization nozzles for use herein are available, for example, from Fog Atomic Technologies, LLC, Medina, Ohio. In various embodiments, an atomization nozzle may comprise a heat conducting portion surrounding the orifice leading to the nozzle tip, and a heating jacket around the heat conducting portion. The nozzle may be a hollow cone nozzle having an orifice diameter of from about 0.1 μm to about 100 μm. The spray pattern of the atomized droplets 5 may be a cone (10°-90°, for example), or a flat fan (60°-90°, for example), or any other desired spray pattern. Spray patterns may be selected such that the atomized droplets impinge on various target surfaces, e.g., the inside walls of the atomization chamber that may be heated to vaporize the volatile components in the droplets.

Ultrasonic nozzles (i.e., ultrasonic nebulizers) operate by converting high frequency sound waves into mechanical energy that is transferred into the liquid extraction mixture, creating standing waves that break the liquid into fine droplets. Typically, ultrasonic nozzles do not require a pressurized liquid feed, although they do require an electrical supply. Therefore, when the decarboxylation and solvent recovery apparatus comprises an atomization chamber fitted only with ultrasonic nozzles, P1 may be simply ambient pressure, and the pressurized gas feed (N₂, etc.) may be eliminated. For an ultrasonic nozzle, the liquid extraction mixture may be gravity fed to the nozzle, or at least pumped to the nozzle if gravity feed is not practical. Ultrasonic nozzles for use herein are commercially available from any number of suppliers.

Using either an atomization nozzle with pressurized liquid feed, or an ultrasonic nozzle with gravity feed, the target average particle size of the atomized droplets thus produced is from about 0.1 μm to about 100 μm. Other particle sizes for the atomized droplets outside of this range may be found better suited for certain fractionation processes and/or to facilitate various chemical reactions. For the standard atomization nozzles, orifice diameter and/or inlet pressure P1 may be adjusted so that the resulting atomized particles have an average particle size of from about 0.1 μm to about 100 μm. For the ultrasonic nozzles, the frequency of the nozzle may be adjusted (e.g., from about 10 to about 200 KHz) so that the resulting atomized particles have an average particle size of from about 0.1 μm to about 100 μm.

The atomized droplets may be further manipulated by impinging a disruptor gas into the atomized mist within the atomization chamber. As shown schematically in FIG. 3, a process gas tank 24 may contain a disruptor gas that is fed through line 26 directly into the atomized mist within the chamber. The position of the outlet of the disruptor gas in the atomization chamber may be any distance necessary from the outlet of the atomization or ultrasonic nozzle 4 in order to achieve a desired result. In various embodiments, a disruptor gas may comprise CO₂. A disruptor gas may be used to further minimize the diameter of atomized droplets through high velocity impingement on, and/or agitation of, the atomized droplets to prevent or at least mitigate agglomeration (coalescing) of atomized droplets. In other words, impingement of a disruptor gas onto atomized particles is a way to keep the droplets dispersed and prevent them from coalescing into larger droplets. In various embodiments, the disruptor gas will travel through the apparatus 1 where it can be captured and recycled.

In other embodiments, atomized droplets within the atomization chamber may be disrupted by something other than a disruptor gas. For example, a sound transducer may be positioned in the atomized mist to agitate the atomized droplets and keep them from coalescing into larger droplets. In some instances, an ultrasonic transducer, e.g., from the medical profession, may be used.

### C. First and Second Condensers:

In various embodiments, a decarboxylation and solvent recovery apparatus in accordance with the present disclosure comprises a first condenser 9 and a second condenser 13 arranged in series from the outlet 8 of the atomization chamber and coupled in fluidic communication. In various embodiments, there may be more than two condensers within a series of condensers, fluidically connected in a chain of condensers. Herein, condensers may be heated, held near ambient, or chilled as needed so that certain desired constituents of the liquid extraction mixture, and chemical reaction products therefrom, are captured and other desired constituents of the liquid extraction mixture, and chemical reaction products therefrom move further downstream in the fractionation process. In the simplest sense, a condenser comprises an inlet and outlet, a drain (i.e., a second outlet), an interior space, and a heating or cooling jacket that is external or internal to the outer surface of the condenser, or both. Complex industrial condensers may further comprise internal baffles or other internal structural features. In various embodiments, a first condenser and a second condenser may be of identical design, or different structural designs as needed.

With reference still to FIG. 3, a first condenser 9 is fluidically connected between the outlet 8 of the atomization chamber 6 and the inlet 14 of a second condenser 13. The first condenser 9 comprises an inlet 10, an outlet 11 and a drain 12. The inlet 10 allows for entry of the atomized liquid extraction mixture 5. Whatever material doesn't condense within the first condenser 9 exits out through outlet 11 and into the second condenser 13. What does condense in the first condenser 9 may be collected at the drain 12. This material may be referred to as the “primary distillate.” In various embodiments, the primary distillate collected at drain 12 comprises cannabinoids. In various examples, at least one or more of the cannabinoids collected at drain 12 has undergone a decarboxylation reaction sometime in the atomization process, either while passing through the atomization nozzle 4 and/or in the atomization chamber 6, such as by contacting heated walls of the chamber. In various embodiments, the material exiting the first condenser 9 at outlet 11 and entering the second condenser 13 at through inlet 14 comprises mostly extraction solvent, e.g., ethanol. This vapor may also comprise other volatile materials, such as esters, extracted from the plant material and present in the starting liquid extraction mixture. In various embodiments, the material exiting the first condenser 9 at outlet 11 and entering the second condenser 13 at through inlet 14 may comprise both vaporized constituents and atomized constituents. As mentioned, the purity of the cannabinoid mixture collected at drain 12 may be analyzed. If the primary distillate is contaminated with an unacceptable amount of extraction solvent or other volatile materials, and/or if the desired cannabinoids in the primary distillate have not undergone the desired decarboxylation reactions, primary distillate can be recirculated to the supply tank 2 through a recirculation fluidic connection pathway 23. Aspects of this recirculation may be automated. For example, the primary distillate may be optically analyzed, the results input to a computer, and the computer switching the necessary valve(s) to either turn on, turn off, or adjust the recirculation flow of primary distillate back to the liquid feed tank 2. In this way, the liquid extraction mixture in the liquid feed tank 2 may be enriched by the recycling of primary distillate that is rich in cannabinoid oils and low in volatile extraction solvent.

In various embodiments, the primary distillate, rich in cannabinoids, may be separately subjected to a secondary fractionation process that is independent of the apparatus depicted in FIG. 3. For example, the primary distillate may be subjected to column chromatography or thin layer chromatography to separate the various cannabinoids in the mixture. If necessary, some cannabinoids may be returned to the liquid extraction mixture in liquid feed tank 2, particularly if a cannabinoid of interest still remains as an undesired carboxylic acid and has not undergone decarboxylation.

In various embodiments, the temperature CT1 within the first condenser 9 may be from about −100° C. to about 100° C. In other embodiments, the temperature CT1 within the first condenser 9 may be from about −80° C. to about 0° C. These condenser temperature ranges are not meant to be limiting in any sense. CT1 temperatures outside these ranges may be found better suited for condensing certain materials.

In various embodiments, the condensation process within first condenser 9 may further comprise a partial falling film or a rising film trap.

With continued reference to FIG. 3, a second condenser 13 is fluidically connected between the outlet 11 of the first condenser 9 and the inlet 18 of the vacuum pump 17. The second condenser may be structurally the same as the first condenser 9, or it may comprise other internal or external features. The second condenser 13 comprises an inlet 14, an outlet 15 and a drain 16. The inlet 14 allows for entry of the vapors and aerosols not condensed as primary distillate in the first condenser 9. Whatever material doesn't condense within the second condenser 13 exits out through outlet 15, through the vacuum pump 17, and into the surroundings. In various embodiments, the vapors exiting the vacuum pump can be collected, or at least scrubbed as necessary in an abatement process to meet local air emission standards and regulations. In various embodiments, the vapors exiting at 19 from the vacuum pump 17 comprise CO₂.

The material that does condense in the second condenser 13 may be collected at drain 16. This material may be referred to as the “secondary distillate.” In various embodiments, the secondary distillate collected at drain 16 comprises the recovered extraction solvent. In various embodiments, the secondary distillate may be further processed, such as through a secondary fractionation process, which can be on-site or even off site from the decarboxylation and solvent recovery process. For example, secondary distillate may be shipped in tanker trucks to a third party who performs the secondary fractionation process on the large volumes of recycles solvent.

If the secondary distillate is deemed contaminated with unacceptable levels of cannabinoids, other natural products, or other non-volatile materials, secondary distillate can be recirculated to the supply tank 2 through a recirculation fluidic connection pathway 23, and/or secondary distillate can be recirculated to the first condenser 9 through a recirculation fluidic connection pathway 23. Aspects of these two recirculation processes may be automated. For example, the secondary distillate may be analyzed, such as in a continuous process through a flow cell, the results input to a computer, and the computer switching the necessary valve(s) to either turn on, turn off, or adjust the recirculation flow of secondary distillate back to the liquid feed tank 2 or back into the first condenser 9.

In various embodiments, the temperature CT2 within the second condenser 13 may be from about −100° C. to about 100° C. In other embodiments, the temperature CT2 within the second condenser 13 may be from about −80° C. to about 0° C. These condenser temperature ranges are not meant to be limiting in any sense. CT2 temperatures outside these ranges may be found better suited for condensing certain materials.

In various embodiments, the condensation process within second condenser 13 may further comprise a partial falling film or a rising film trap.

In various embodiments, T1>T2>CT1=CT2. In other embodiments, T1>T2>CT1>CT2.

### D. Vacuum Pump:

In various embodiments, a decarboxylation and solvent recovery apparatus in accordance with the present disclosure comprises a vacuum pump to control pressures throughout the system and to pump the vapors and aerosols through the apparatus. In various embodiments, the vacuum pump may be used to ensure that P2 in the atomization chamber is at least somewhat less than P1, the feed pressure to the atomization nozzle. As illustrated in FIG. 3, a vacuum pump 17 is fluidically connected to the outlet of the second condenser 13. Since the atomization chamber 6, first condenser 9 and second condenser 13 are connected in series in fluidic communication, and connected to the vacuum pump 17, the vacuum pump 17 controls pressure throughout the three components upstream in the apparatus. The outlet 19 from vacuum pump 17 provides the exit for the CO₂ liberated in the decarboxylation reactions in addition to expulsion of any feed gas or disruptor gas that may also comprise CO₂. Other volatile material, such as residual solvent, exiting at outlet 19 may be condensed and recycled. In various embodiments, a vacuum regulator between the outlet 15 of the second condenser 13 and the inlet 18 of the vacuum pump 17 can be used to maintain a target pressure within the atomization chamber 6, first condenser 9 and second condenser 13 of from about 1 bar (750 mmHg) down to about 0.00013 bar (0.1 mmHg). The vacuum pump may be used to control pressures inside the atomization chamber 6, first condenser 9 and second condenser 13 outside this exemplary range. Any type of vacuum pump can be used for this purpose.

### Decarboxylation of Cannabinoid Carboxylic Acids in the Atomization Process

As discussed, certain cannabinoids are extracted from plant material as carboxylic acids, and it is desirable to promote decarboxylation of the carboxylic acids to the commercially useful cannabinoids. For example, conversion of THC-acid to THC and conversion of CBD-acid to CBD is desirable. It has now been unexpectedly discovered that the burst atomization process as disclosed herein provides concomitant decarboxylation of cannabinoid carboxylic acids. That is, in various embodiments, a liquid extraction mixture comprises cannabinoid acids whereas the primary distillate collected comprises decarboxylated cannabinoids.

Both THC-acid and CBD-acid are aryl carboxylic acids having a hydroxyl moiety α- to the —CO₂H substituent. Typically, decarboxylation of aryl carboxylic acids requires some sort of catalysis, such as with copper/quinoline or a strong acid. It is believed that decarboxylation of an aryl acid such as THC-acid or CBD-acid upon atomization across a pressure gradient is heretofore unknown. The extent (% yield) of decarboxylation, and the control of decarboxylation a particular cannabinoid acid in preference to another, are optimized by changing the atomization variables, that is, P1, T1, P2 and T2. It remains possible that catalysts can be added to the interior walls of the atomization chamber, and/or to the atomization or ultrasonic nozzle. For example, brass components may provide small amounts of copper that can catalyze the desired decarboxylation reactions occurring during atomization of cannabinoid acids. In some examples, a catalyst to promote decarboxylation may be added to the liquid extraction mixture prior to atomization.

In various embodiments, a liquid extraction mixture comprises THC-acid. When the liquid extraction mixture is processed through the decarboxylation and solvent recovery apparatus (e.g., FIG. 3), the primary distillate comprises THC. In various embodiments, decarboxylation of THC-acid occurs during atomization of the liquid extraction mixture comprising the THC-acid.

In various embodiments, a liquid extraction mixture comprises CBD-acid. When the liquid extraction mixture is processed through the decarboxylation and solvent recovery apparatus (e.g., FIG. 3), the primary distillate comprises CBD. In various embodiments, decarboxylation of CBD-acid occurs during atomization of the liquid extraction mixture comprising the CBD-acid.

In various embodiments, a liquid extraction mixture comprises both THC-acid and CBD-acid. When the liquid extraction mixture is processed through the decarboxylation and solvent recovery apparatus (e.g., FIG. 3), the primary distillate comprises both THC and CBD. In various embodiments, decarboxylation of THC-acid and CBD-acid occurs during atomization of the liquid extraction mixture comprising the THC-acid and the CBD-acid.

Methods for Decarboxylating Cannabinoid Acids and Recovering Extraction Solvent from Liquid Extraction Mixtures Comprising Cannabinoid Acids and an Extraction Solvent

In various embodiments, a decarboxylation and solvent recovery apparatus such as depicted schematically in FIG. 1 is used to provide both decarboxylation of cannabinoid acids and recovery of the extraction solvent used to extract the cannabinoid acids from plant material.

In various embodiments, a method for simultaneously decarboxylating cannabinoid acids and recovering extraction solvent present in a liquid extraction mixture comprising the cannabinoid acids and the extraction solvent, the method comprising: atomizing the liquid extraction mixture into atomized particles; conveying the atomized particles into a first condenser wherein decarboxylated cannabinoids are collected as a primary condensate; and capturing vaporized extraction solvent by condensation in a second condenser. In various embodiments, atomizing comprises an atomizing nozzle. In various embodiments, atomizing comprises an ultrasonic nozzle. In various embodiments, atomization provides atomized particles having an average particle size of from about 0.1 μm to about 100 μm.

In various embodiments, a method for simultaneously decarboxylating cannabinoid acids and recovering extraction solvent present in a liquid extraction mixture comprising the cannabinoid acids and the extraction solvent, the method comprising: atomizing the liquid extraction mixture at temperature T1 and pressure P1 into atomized particles within an atomization chamber maintained at temperature T2 and pressure P2; conveying the atomized particles under vacuum into a first condenser maintained at a temperature CT1, wherein decarboxylated cannabinoids are collected as a primary condensate from the primary condenser; and conveying uncondensed vapor from the first condenser into a second condenser maintained at a temperature CT2, wherein recovered extraction solvent is collected as a secondary condensate from the second condenser. In various embodiments, P1>P2 and T1>T2. In various embodiments, atomizing comprises an atomization nozzle fitted to an inlet of the atomization chamber. In various embodiments, the atomized particles have an average particle size of from about 0.1 μm to about 100 μm. In various embodiments, P1 is from about 1.02 bar (765 mmHg) to about 100 bar (75,000 mmHg). In various embodiments, T1 is from about 23° C. to about 500° C. In various embodiments, P2 is from about 1 bar (750 mmHg) down to about 0.00013 bar (0.1 mmHg). In various embodiments, T2 is from about 23° C. to about 500° C. In various embodiments, CT1 and CT2 are independently from about −100° C. to about 100° C. None of these exemplary ranges are meant to be limiting in any way, and conditions outside these ranges may be found more suitable for certain fractionations.

In various embodiments, a method for simultaneously decarboxylating cannabinoid acids and recovering extraction solvent present in a liquid extraction mixture comprising the cannabinoid acids and the extraction solvent, the method comprising: atomizing the liquid extraction mixture at temperature T1 and pressure P1 into atomized particles within an atomization chamber maintained at temperature T2 and pressure P2; conveying the atomized particles under vacuum into a first condenser maintained at a temperature CT1, wherein decarboxylated cannabinoids are collected as a primary condensate from the primary condenser; and conveying uncondensed extraction solvent vapor from the first condenser into a second condenser maintained at a temperature CT2, wherein recovered extraction solvent is collected as a secondary condensate from the second condenser. In various embodiments, P1>P2 and T1>T2. In various embodiments, atomizing comprises an ultrasonic nozzle fitted to an inlet of the atomization chamber and powered by an electrical supply. In various embodiments, the atomized particles have an average particle size of from about 0.1 μm to about 100 μm. In various embodiments, P1 is about atmospheric pressure. In various embodiments, T1 is from about 23° C. to about 500° C. In various embodiments, P2 is from about 1 bar (750 mmHg) down to about 0.00013 bar (0.1 mmHg). In various embodiments, T2 is from about 23° C. to about 500°. In various embodiments, CT1 and CT2 are independently from about −100° C. to about 100°. In various embodiments, the ultrasonic nozzle is adjusted to an operating frequency of from about 10 to about 200 KHz, such as to achieve particle sizes of from about 0.1 μm to about 100 μm.

Systems for Decarboxylating Cannabinoid Acids and Recovering Extraction Solvent from Liquid Extraction Mixtures Comprising Cannabinoid Acids and an Extraction Solvent

In various embodiments, a fractionation system comprises: a liquid feed tank 2 containing a liquid extraction mixture comprising cannabinoid carboxylic acids and extraction solvent; an atomization chamber 6 having an inlet 7 and outlet 8, the inlet 7 fluidically connected to the liquid feed tank 2; an atomization nozzle 4 fitted within inlet 7; a first condenser 9 maintained at a temperature of CT1 and having an inlet 10 and outlet 11, the inlet 10 fluidically connected to the outlet 8; a second condenser 13 maintained at a temperature of CT2 and having an inlet 14 and an outlet 15, the inlet 14 fluidically connected to the outlet 11; a vacuum pump 17 having an inlet 18 and an outlet 19, the inlet 18 fluidically connected to the outlet 15; optionally a fluidic recirculation pathway from the first condenser 9 to the liquid feed tank 2; optionally a fluidic recirculation pathway from the second condenser 13 to the liquid feed tank 2; and optionally a fluidic recirculation pathway from the second condenser 13 to the first condenser 9, wherein the liquid extraction mixture is supplied from the liquid feed tank 2 to the atomization nozzle 4 at pressure P1 and temperature T1, whereby the liquid extraction mixture is atomized into the atomization chamber 6 as fine particles with concomitant decarboxylation of the cannabinoid carboxylic acids to their corresponding decarboxylated cannabinoids; and wherein the corresponding decarboxylated cannabinoids are captured as a first distillate from the first condenser 9; the extraction solvent is captured as a second distillate from the second condenser 13; and carbon dioxide liberated from decarboxylation of the cannabinoid carboxylic acids is emitted from the outlet 19 of the vacuum pump 17. In various embodiments, the atomization nozzle may be substituted with an ultrasonic nozzle (nebulizer) that does not require a pressured feed but can deliver atomized particles into the atomization chamber maintained under a vacuum. The ultrasonic nozzle is electrically connected and frequency adjustable to obtain the desired average particle size of the atomized droplets.

### Additional Aspects

In various embodiments, a burst atomization fractionation apparatus is disclosed. The apparatus comprises: an atomization chamber having interior walls defining an interior at a pressure P2, the chamber fitted with an atomization nozzle fluidically connecting a liquid feed inlet to the interior, the atomization nozzle configured to atomize a liquid from the liquid feed inlet into the interior in the form of fine particles; a liquid feed tank containing a liquid mixture fluidically connected to the liquid feed inlet, the liquid mixture comprises at least two components selected from volatiles, essentially non-volatiles, and non-volatile solutes; a vacuum pump fluidically connecting the interior through a vapor outlet to a space outside the chamber; and a liquid outlet fluidically connecting the interior to a space outside the chamber; wherein at least a portion of an interior wall of the chamber is at a temperature T2; and wherein the liquid mixture at the liquid feed inlet is at a pressure P1 and at a temperature T1, wherein P2<P1 and T2<T1.

In various embodiments, the apparatus further comprises a pressurized tank of process gas to provide the liquid mixture to the atomization nozzle at the pressure P1.

In various embodiments, the process gas comprises any one of CO₂, nitrogen or argon.

In various embodiments, the apparatus further comprises a combination of heating and cooling coils or heating and cooling jackets on or in proximity to the atomization chamber.

In various embodiments, the atomization nozzle is configured to provide a distribution pattern of droplets that impinge upon at least a portion of an interior wall.

In various embodiments, the atomization nozzle is configured to provide a distribution pattern of droplets that impinge upon at least a portion of at least one of a shelf, tray or baffle positioned within the interior of the atomization chamber.

In various embodiments, the liquid mixture for fractionation in the burst atomization fractionation apparatus consists essentially of salt water, and wherein the shelf, tray or baffle configured inside the atomization chamber is configured to collect salt.

In various embodiments, the apparatus further comprises a cold trap disposed in the vapor outlet between the atomization chamber and the vacuum pump, wherein the cold trap is maintained at <0° C., such as with dry ice.

In various embodiments, the cold trap is maintained at a temperature sufficient to condense at least one volatile solvent.

In various embodiments, the liquid mixture for fractionation in the burst atomization fractionation apparatus comprises a mixture of volatile solvents.

In various embodiments, the liquid mixture for fractionation in the burst atomization fractionation apparatus comprises a mixture of at least one volatile solvent, such as an extraction solvent, and at least one essentially non-volatile natural product.

In various embodiments, the at least one essentially non-volatile natural product comprises cannabinoid oils, terpenes, sesquiterpenes, diterpenes, triterpenes, or fatty acids. Cannabinoid oils may further comprise cannabinoid carboxylic acids such as TCH-acid or CBD-acid.

In various embodiments, the liquid mixture for fractionation in the burst atomization fractionation apparatus comprises used engine oil.

In various embodiments, the apparatus further comprises at least one condenser fluidically connected on the vapor outlet between the atomization chamber and the vacuum pump, such that vapors from the liquid mixture are at least partially condensed in the at least one condenser.

In various embodiments of the present disclosure, a method of fractionating a liquid mixture is described. The method comprises: atomizing the liquid mixture from a temperature T1 and a pressure P1 into fine particles dispersed within an atomization chamber, the atomization chamber having interior walls defining an interior, the interior configured to a pressure P2; impinging at least a portion of the atomized fine particles onto a portion of an interior wall configured to a temperature T2, wherein at least one component of the liquid mixture is recovered as a condensate removed from the atomization chamber; conveying at least a portion of the atomized fine particles through a vapor outlet from the interior of the chamber to a vacuum pump outside the chamber; and condensing at least one component of the liquid mixture outside the chamber; wherein P2<P1 and T2<T1.

In various embodiments, at least one component of the liquid mixture is condensed in a cold trap disposed in the vapor outlet between the atomization chamber and the vacuum pump.

In various embodiments, at least one component of the atomized liquid mixture is at least partially condensed in a condenser fluidically connected on the vapor outlet between the atomization chamber and the vacuum pump, and wherein the at least one component is removed and recovered from the condenser.

In various embodiments of the method, a pressurized tank of process gas provides the liquid mixture to the atomization nozzle at the pressure P1.

In various embodiments, the process gas comprises any one of CO₂, nitrogen or argon.

In various embodiments, a combination of heating and cooling coils or heating and cooling jackets in contact with the atomization chamber maintain at least a portion of an interior wall of the atomization chamber at the temperature T2.

In various embodiments, the liquid mixture for fractionation by the method comprises a mixture of volatile solvents.

In various embodiments, the liquid mixture for fractionation by the method comprises a mixture of at least one volatile solvent and at least one essentially non-volatile natural product.

In various embodiments, the at least one essentially non-volatile natural product comprises cannabinoid oils, terpenes, sesquiterpenes, diterpenes, triterpenes, or fatty acids. The cannabinoid oils may further comprise cannabinoid carboxylic acids.

In various embodiments, the liquid mixture for fractionation by the method comprises used engine oil.

In various embodiments, the liquid mixture for fractionation by the method comprises salt water, and thus the method comprises a desalination process.

In various embodiments, and with reference to the element numbering in FIG. 3, a fractionation apparatus is described. The fractionation apparatus comprises: an atomization chamber 6 maintained at a pressure P2 and temperature T2 and having an inlet 7 and outlet 8; an atomization nozzle 4 or ultrasonic nozzle 4 fitted within inlet 7; a first condenser 9 maintained at a temperature of CT1 and having an inlet 10 and outlet 11, the inlet 10 fluidically connected to the outlet 8; a second condenser 13 maintained at a temperature of CT2 and having an inlet 14 and an outlet 15, the inlet 14 fluidically connected to the outlet 11; and a vacuum pump 17 having an inlet 18 and an outlet 19, the inlet 18 fluidically connected to the outlet 15, wherein a liquid mixture comprising an essentially non-volatile substance and volatile solvent is supplied from a liquid feed tank 2 to the atomization nozzle 4 or ultrasonic nozzle 4 at pressure P1 and temperature T1, whereby the liquid mixture is atomized into the atomization chamber 6 as fine particles; and wherein the essentially non-volatile substance is captured as a first distillate from the first condenser 9 and the volatile solvent is captured as a second distillate from the second condenser 13.

In various embodiments, the apparatus further comprises recirculation pathways for any one of recycling of the first condensate from the first condenser 9 to the liquid feed tank 2, recycling of the second condensate from the second condenser 13 to the liquid feed tank 2, and recycling of the second condensate from the second condenser 13 to the first condenser 9.

In various embodiments, the apparatus further comprises a pressurized tank of gas to provide the liquid mixture at P1 to the atomization nozzle 4.

In various embodiments, the gas comprises any one of CO₂, nitrogen or argon.

In various embodiments, any one of the first condenser 9 and the second condenser 13 further comprises partial falling film or rising film trap fractionation.

In various embodiments, the essentially non-volatile substance is selected from the group consisting of THC-acid, CBD-acid, or mixtures thereof.

In various embodiments, the first distillate further comprises corresponding decarboxylated cannabinoids produced by decarboxylation of the cannabinoid carboxylic acids during the atomization of a liquid mixture comprising cannabinoid carboxylic acids.

In various embodiments, the first distillate comprises corresponding decarboxylated cannabinoids selected from the group consisting of THC, CBD, and mixtures thereof.

In various embodiments, the atomized fine particles have an average particle size of from about 0.1 μm to about 100 μm.

In various embodiments, P1>P2.

In various embodiments, T1>T2.

In various embodiments, T1>T2>CT1>CT2.

In various embodiments, the apparatus further comprises a process gas tank 24 containing at least one of a feed gas or disruptor gas fluidically connected to at least one of the nozzle 4 and the atomization chamber.

In various embodiments, the disruptor gas is fed into the atomization chamber whereby the disruptor gas impinges upon the atomized fine droplets.

In various embodiments, the volatile solvent comprises a low molecular weight alcohol selected from the group consisting of methanol, ethanol, n-propanol, isopropanol, n-butanol, and mixtures thereof.

In various embodiments, the essentially non-volatile substance comprises an essential oil.

In various embodiments, the essentially non-volatile substance comprises at least one of a cannabinoid, alkaloid, terpene, flavonoid or polysaccharide.

In various embodiments, the liquid mixture comprises an ethanol extract of cannabinoid carboxylic acids.

In various embodiments, a method for the simultaneous decarboxylation of cannabinoid carboxylic acids and the recovery of extraction solvent from a liquid extraction mixture comprising the cannabinoid carboxylic acids and the extraction solvent is described.

The method comprises: atomizing the liquid extraction mixture at temperature T1 and pressure P1 into fine particles within an atomization chamber maintained at temperature T2 and pressure P2; conveying the fine particles under vacuum into a first condenser maintained at a temperature CT1, wherein decarboxylated cannabinoids corresponding to the cannabinoid carboxylic acids are collected as a primary condensate from the primary condenser; and conveying uncondensed extraction solvent vapor exiting from the first condenser into a second condenser maintained at a temperature CT2, wherein recovered extraction solvent is collected as a secondary condensate from the second condenser.

In various embodiments, the cannabinoid carboxylic acids are selected from the group consisting of THC-acid, CBD-acid, or mixtures thereof.

In various embodiments, the corresponding decarboxylated cannabinoids are selected from the group consisting of THC, CBD, and mixtures thereof.

In various embodiments, the atomized fine particles have an average particle size of from about 0.1 μm to about 100 μm.

In various embodiments, P1>P2.

In various embodiments, T1>T2.

In various embodiments, T1>T2>CT1>CT2.

In various embodiments, the corresponding decarboxylated cannabinoids are produced by decarboxylation of the cannabinoid carboxylic acids during the atomization of the liquid extract mixture comprising the cannabinoid carboxylic acids.

In various embodiments, and with reference to the elements in FIG. 3, a fractionation system comprises: a liquid feed tank 2 containing a liquid extraction mixture comprising an extraction solvent and a natural product extracted and dissolved therein; an atomization chamber 6 having an inlet 7 and outlet 8, the inlet 7 fluidically connected to the liquid feed tank 2; an atomization nozzle 4 fitted within inlet 7; a first condenser 9 maintained at a temperature of CT1 and having an inlet 10 and outlet 11, the inlet 10 fluidically connected to the outlet 8; a second condenser 13 maintained at a temperature of CT2 and having an inlet 14 and an outlet 15, the inlet 14 fluidically connected to the outlet 11; a vacuum pump 17 having an inlet 18 and an outlet 19, the inlet 18 fluidically connected to the outlet 15; optionally a fluidic recirculation pathway from the first condenser 9 to the liquid feed tank 2; optionally a fluidic recirculation pathway from the second condenser 13 to the liquid feed tank 2; and optionally a fluidic recirculation pathway from the second condenser 13 to the first condenser 9, wherein the liquid extraction mixture is supplied from the liquid feed tank 2 to the atomization nozzle 4 at pressure P1 and temperature T1, whereby the liquid extraction mixture is atomized into the atomization chamber 6 as fine particles; and wherein the natural product is captured as a first distillate from the first condenser 9 and the extraction solvent is captured as a second distillate from the second condenser 13.

In various embodiments, the natural product in the liquid extraction mixture undergoes a chemical reaction during atomization.

In various embodiments, the natural product is a cannabinoid carboxylic acid and the chemical reaction comprises decarboxylation.

In various embodiments, the natural product is selected from the group consisting of THC-acid, CBD-acid, and mixtures thereof.

In various embodiments, the first distillate comprises a chemically transformed solute selected from the group consisting of THC, CBD, and mixtures thereof.

In various embodiments, the system further comprises a pressurized tank of feed gas to provide the liquid extraction mixture at P1 to the atomization nozzle 4.

In various embodiments, the feed gas comprises at least one of CO₂, nitrogen or argon.

In various embodiments, any one of the first condenser 9 and the second condenser 13 further comprises partial falling film or rising film trap fractionation.

In various embodiments, the atomized fine particles have an average particle size of from about 0.1 μm to about 100 μm.

In various embodiments, P1>P2.

In various embodiments, T1>T2.

In various embodiments, T1>T2>CT1>CT2.

In various embodiments, the system further comprises a process gas tank 24 containing at least one of a feed gas or disruptor gas fluidically connected to at least one of the nozzle 4 and the atomization chamber.

In various embodiments, the disruptor gas is fed into the atomization chamber whereby the disruptor gas impinges upon the atomized fine droplets.

In various embodiments, the extraction solvent comprises a low molecular weight alcohol selected from the group consisting of methanol, ethanol, n-propanol, isopropanol, n-butanol, and mixtures thereof.

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

We claim:

1. A burst atomization fractionation apparatus comprising:
an atomization chamber having interior walls defining an interior, the atomization chamber fitted with an atomizer fluidically connecting a liquid feed inlet to the interior of the atomization chamber, the atomizer configured to atomize a liquid mixture from the liquid feed inlet into the interior of the atomization chamber in the form of atomized fine particles;
a vacuum pump fluidically connecting the interior of the atomization chamber through a vapor outlet to an environment outside the atomization chamber; and
a liquid outlet fluidically connecting the interior of the atomization chamber to an environment outside the atomization chamber.

2. The burst atomization fractionation apparatus of claim 1, wherein the atomizer comprises an atomization nozzle or an ultrasonic nebulizer.

3. The burst atomization fractionation apparatus of claim 1, further comprising a liquid feed tank fluidically connected to the liquid feed inlet, the liquid feed tank configured to feed a liquid mixture contained in the liquid feed tank to the liquid feed inlet.

4. The burst atomization fractionation apparatus of claim 3, further comprising a pressurized tank of process gas fluidically connected to at least one of the liquid feed tank or the liquid feed inlet, the pressurized tank of process gas configured to pressurize a liquid mixture in the liquid feed tank or present at the atomizer.

5. The burst atomization fractionation apparatus of claim 1, wherein the atomization chamber further comprises a combination of heating and cooling coils or jackets configured to heat and cool the interior of the atomization chamber.

6. The burst atomization fractionation apparatus of claim 1, wherein the atomizer is configured to directionally impinge at least a portion of the atomized fine particles upon at least a portion of the interior walls of the atomization chamber.

7. The burst atomization fractionation apparatus of claim 1, further comprising at least one of a shelf, tray or baffle positioned within the interior of the atomization chamber.

8. The burst atomization fractionation apparatus of claim 1, further comprising at least one condenser, each condenser comprising an interior fluidically connected in series between the atomization chamber and the vacuum pump, a liquid outlet connecting the interior of the condenser to an environment outside the condenser, and a combination of heating and cooling coils or jackets configured to heat and cool the interior of the condenser.

9. The burst atomization fractionation apparatus of claim 1, further comprising a pressurized tank of disrupter gas fluidically connected through an injector line to the interior of the atomization chamber, the injector line configured to inject disrupter gas into the atomized fine particles.

10. A method of fractionating a liquid mixture comprising at least first and second components, the method comprising:
atomizing the liquid mixture from a temperature T1 and a pressure P1 into an interior of an atomization chamber in the form of atomized fine particles, the atomization chamber having interior walls defining the interior, the interior configured at a temperature T2 and a pressure P2;
recovering at least the first component of the liquid mixture as a liquid condensate from the interior of the atomization chamber;
recovering at least the second component of the liquid mixture as a vapor exiting from the interior of the atomization chamber;
wherein P2<P1 and T2<T1.

11. The method of claim 10, wherein the atomizing comprises atomization of the liquid mixture through an atomization nozzle or an ultrasonic nebulizer.

12. The method of claim 11, wherein a pressurized tank of process gas provides the liquid mixture to the atomization nozzle at the pressure P1.

13. The method of claim 10, wherein the atomization chamber further comprises a combination of heating and cooling coils or jackets configured to maintain at least a portion of the interior walls of the atomization chamber at temperature T2 so as to promote condensation of at least the first component of the liquid mixture.

14. The method of claim 10, wherein a vacuum pump fluidically connected to the interior of the atomization chamber maintains the interior of the atomization chamber at pressure P2.

15. The method of claim 14, wherein at least one condenser fluidically connected between the interior of the atomization chamber and the vacuum pump condenses at least the second component of the liquid mixture exiting from the atomization chamber as a vapor into a liquid condensate.

16. The method of claim 10, wherein the liquid mixture comprises at least one of a mixture of volatiles, a mixture of essentially non-volatiles and volatiles, or a mixture of non-volatile solutes and volatiles.

17. The method of claim 10, wherein the liquid mixture comprises (i) at least one essentially non-volatile natural product in at least one extraction solvent; (ii) used engine oil; or (iii) salt water.

18. The method of claim 17, wherein the at least one essentially non-volatile natural product comprises a cannabinoid carboxylic acid.

19. The method of claim 18, wherein the first component of the liquid mixture recovered as a liquid condensate comprises a decarboxylated cannabinoid produced by decarboxylation of the cannabinoid carboxylic acid during the atomization of the liquid mixture.

20. The method of claim 10, wherein the liquid mixture comprises at least one cannabinoid carboxylic acid and ethanol.
